(19) United States
(12) Patent Application Publication
(10) Pub. No.: US 2024/0207324 A1
(43) Pub. Date: Jun. 27, 2024

(54) METHOD FOR QUALITY CONTROL AND ENRICHMENT OF HUMAN DOPAMINERGIC NEURAL PRECURSOR CELLS

(71) Applicant: SHANGHAI UNIXELL BIOTECHNOLOGY CO., LTD., Shanghai (CN)

(72) Inventors: Yuejun CHEN, Shanghai (CN); Peibo XU, Shanghai (CN); Ziyan WU, Shanghai (CN)

(73) Assignee: SHANGHAI UNIXELL BIOTECHNOLOGY CO., LTD., Shanghai (CN)

(21) Appl. No.: 18/556,510

(22) PCT Filed: Apr. 20, 2022
(86) PCT No.: PCT/CN2022/088016

§ 371 Date: Oct. 20, 2023

(30) Foreign Application Priority Data
Apr. 21, 2021 (CN) 202110429176.9

(51) Int. Cl. A61K 35/30; A61P 25/16; C12N 5/0793; C12Q 1/6881

(52) U.S. Cl. CPC A61K 35/30; A61P 25/16; C12N 5/0619; C12Q 1/6881; C12N 2506/45; C12Q 2600/158

(57) ABSTRACT

Provided are a method for identifying, separating and/or enriching dopaminergic neural precursor cells, the method comprising: detecting whether a candidate cell possesses one or more among the following features: CLSTN2+, PTPRO+, NTRK3+, FLRT2+, KITLG+, CD83+, and/or LMX1A+EN1+, and cell products obtained according to the method. Also provided is a method for evaluating a cell product and optimizing a cell product preparation process. The product and methods can be used to treat diseases or disorders of the nervous system.

### TECHNICAL FIELD

The present application relates to the field of biomedicine, and in particular to a method for quality control and enrichment of human dopaminergic neural precursor cells using a novel cell surface marker or a transcription factor combination.

### BACKGROUND OF THE INVENTION

Parkinson's disease (PD) is the second most common degenerative disease occurred to the central nervous system, following Alzheimer's disease. There is no effective therapeutic regimen for curing the Parkinson's disease clinically, and the existing therapy is mainly symptomatic treatment, including drug treatment represented by levodopa and deep brain stimulation. However, drug treatment is only effective in the early stages, and the deep brain stimulation is only applicable to some patients and may induce side effects such as depression. At present, cell transplantation and replacement therapy (cell therapy) are the most promising treatment for PD. In the cell therapy for PD, human pluripotent stem cells (hPSCs), including human embryonic stem cells (hESCs) and human induced pluripotent stem cells (hPSCs), are typically used to obtain a population of midbrain ventral neural cells including midbrain dopamine (mDA) neural precursor cells through in vitro differentiation, and these cells can be transplanted and then differentiated and matured to obtain grafts containing mDA neurons, thereby rescuing PD patients from motor dysfunction.

However, there are still many problems limiting the wide clinical application of the cell therapy for PD.

First, there is a lack of an effective method for quality control and evaluation of cell preparations. In the cell therapy for PD, it is typical to transplant immature neural precursor (stem) cells rather than terminal neurons, and these immature neural precursor (stem) cells do not express characteristic genes of the terminal neurons prior to transplantation. Also, the differentiation of hPSCs does not result in a single type of neural precursor (stem) cells, but a mixture of multiple types and fates of neural precursor (stem) cells, including mDA neural precursor cells. In clinical treatment, before transplantation to a patient, it is necessary to sample the same batch of cell preparations, transplant it into the brain of a model animal, and perform immunohistochemical detection after at least 3 months, whereby the proportion of target neurons-mDA neurons (which are transplanted into the patient) in the graft can be determined. This evaluation method is time-consuming and laborious.

In the prior art, benefiting from the analysis of the mDA neuronal development process in mouse-based models, the proportion of specific mDA neural precursor cells in hPSCs-derived cell preparations is evaluated by detecting the expression of some key genes (including a marker molecule FOXA2 of the floor plate, a marker molecule OTX2 in the midbrain, a marker molecule LMX1A in the ventral diencephalon-midbrain, and a marker molecule EN1 in the midbrain-hindbrain) in a dopaminergic neuron development process. However, these genes are not only expressed in mDA neural precursor cells, and based on studies, it is found that there is no correlation between the level of expression of these genes in the neural precursor cells and the proportion of mDA neurons after transplantation. In addition, differences in the expression levels of nucleic acid and protein and the consequent difficulty in detection make genes with increased expression on the mDA neural precursor cells not necessarily serve as molecular markers for evaluation or quality control during differentiation. Therefore, although the clinical trials of the cell therapy for PD based on the neural differentiation of hPSCs have been conducted in many countries, there is no method for evaluation and quality control of the cell therapy for PD in the scientific sense.

Secondly, the proportion of target neural cells is low after cell transplantation. In the cell therapy for PD based on the neural differentiation of hPSCs, the proportions of target dopaminergic neurons in the cell preparations obtained by different differentiation methods are low after transplantation, and show a large difference by about 0.3%-20%. At present, there is no effective and specific marker molecules for mDA neural precursor cells. In fact, according to current studies, the mDA marker molecules (for example, CORIN, FOXA2, and LMX1A) are considered to be expressed in many other neural cells in a non-specific way. At present, the use of these marker molecules cannot obviously enrich mDA neurons, or the degree of enrichment is low:

Finally, the cellular composition of the transplanted tissues in the brain is unclear. In the cell therapy for PD based on the neural differentiation of hPSCs, a majority of transplanted blocks are non-dopaminergic neural cells (80%-99.7%), and their type and identity are far from being clearly studied. Meanwhile, non-target neural cells in the transplanted tissues are also a source of potential side effects. Unclear composition of transplanted cells in the brain also makes it difficult to evaluate the safety and long-term potential side effects of the cell therapy for PD.

Therefore, there is an urgent need for methods for quality control, enrichment or evaluation of a cell therapy product for PD.

### SUMMARY OF THE INVENTION

Surprisingly, the inventors of the present application found one or more novel molecular markers of midbrain dopaminergic (mDA) neural precursor cells: CLSTN2⁺, PTPRO⁺, NTRK3⁺, FLRT2⁺, KITLG⁺, CD83⁺, and/or LMX1A⁺EN1⁺. The present application provides methods for use of these molecular markers in identification, isolation and/or enrichment of dopaminergic neural precursor cells or a cell population, as well as a cell product and/or cell preparation prepared by the methods.

In addition, when used in the products and/or methods in the present application, double markers LMX1A+EN1+ can achieve significantly better technical results (for example, significantly better identification, isolation and/or enrichment effects) than the single marker LMX1A+ or the single marker EN1+.

A graft resulting from differentiation of the cell product and/or cell preparation prepared according to the method of the present application shows a high proportion (for example, greater than 30%, 35%, 40%, 45%, 50%, 55%, 60%, 65%, 70%, 75%, 80%, 85%, 95% or higher) of mDA neurons (mainly including substantia nigra dopaminergic neurons).

The graft provided by the present application may has a surprisingly uniform cell composition (for example, available for verification by scRNA-seq analysis and/or histological analysis). It can be seen that this graft may also have a stable and/or predictable outcome after transplantation. The cell composition of the graft provided by the present application is stable and predictable, which greatly improves the effectiveness and safety of the cell therapy for PD. Based on the molecular markers of novel mDA neural precursor cells, the present application provides a series of products and methods for quality control of the production and manufacturing of and for purification of clinical-grade cell therapy preparations for PD, as well as for the predictability of long-term transplantation prognosis, which provides guidance for the clinical application of the cell replacement therapy for the PD.

Meanwhile, the graft resulting from the differentiation of the cell product and/or cell preparation prepared according to the method of the present application can achieve a higher therapeutic efficacy (in other words, showing the advantage of achieving a therapeutic effect only at a required low dose of transplanted cells). For example, the therapeutic effect (for example, the therapeutic effect for PD) of the cell product obtained by a conventional method or a graft thereof can still be achieved by significantly reducing the number of transplanted cells (or example, by at least 50%, at least 60%, at least 70%, at least 80%, at least 90%, at least 95% or higher) in the graft resulting from the differentiation of the cell product and/or cell preparation prepared by the method of the present application.

In addition, the graft resulting from the differentiation of the cell product and/or cell preparation prepared by the method of the present application can provide stronger dopaminergic neuronal innervation.

The present application provides a method for identifying dopaminergic neural precursor cells, comprising: determining whether candidate cells have one or more of the following features: CLSTN2⁺, PTPRO⁺, NTRK3⁺, FLRT2⁺, KITLG⁺, CD83⁺, and/or LMX1A⁺EN1⁺; and identifying cells having the features as the dopaminergic neural precursor cells.

In another aspect, the present application provides a method for preventing, treating, or alleviating neurological diseases or disorders. The method comprises the following steps: identifying whether candidate cells have one or more of the following features: CLSTN2⁺, PTPRO⁺, NTRK3⁺, FLRT2⁺, KITLG⁺, CD83⁺, and/or LMX1A⁺EN1⁺; selecting cells having the features: and administering an effective dose of the cells having the features to a subject in need thereof. In some embodiments, the neurological diseases or disorders comprise Parkinson's disease.

In some embodiments, the candidate cells are neural precursor cells.

In some embodiments, the candidate cells are derived from pluripotent stem cells.

In some embodiments, the candidate cells are derived from human pluripotent stem cells.

In some embodiments, the candidate cells have differentiated in vitro for at least about 10 days.

In some embodiments, the method comprises determining whether the candidate cells have the following feature: CLSTN2⁺.

In some embodiments, the determining comprises directly or indirectly detecting the candidate cells for expression and/or activity level of CLSTN2.

In some embodiments, the expression and/or activity level of CLSTN2 comprises expression and/or activity level of a nucleic acid molecule encoding the CLSTN2, and/or expression and/or activity level of a CLSTN2 protein.

In some embodiments, the detecting comprises modifying the candidate cells.

In some embodiments, the detecting comprises use of marker molecules.

In some embodiments, the marker molecules comprise marker gene proteins, nucleic acids and/or small molecules.

In some embodiments, the marker molecules comprise fluorescent reporter genes.

In some embodiments, the method comprises bringing a reagent capable of specifically binding to the CLSTN2 protein and/or a reagent capable of measuring activity of the CLSTN2 protein into contact with the candidate cells.

In some embodiments, the method comprises bringing primers capable of specifically amplifying a nucleic acid molecule encoding the CLSTN2 and/or probes capable of specifically recognizing the nucleic acid molecule encoding the CLSTN2 into contact with the candidate cells.

In some embodiments, the method comprises determining whether the candidate cells have the following feature: PTPRO⁺.

In some embodiments, the determining comprises directly or indirectly detecting the candidate cells for expression and/or activity level of PTPRO.

In some embodiments, the expression and/or activity level of PTPRO comprises expression and/or activity level of a nucleic acid molecule encoding the PTPRO, and/or expression and/or activity level of a PTPRO protein.

In some embodiments, the indirect detecting comprises modifying the candidate cells.

In some embodiments, the indirect detecting comprises use of marker molecules.

In some embodiments, the marker molecules comprise proteins, nucleic acids and/or small molecules.

In some embodiments, the marker molecules comprise fluorescent reporter genes.

In some embodiments, the method comprises bringing a reagent capable of specifically binding to the PTPRO protein and/or a reagent capable of measuring activity of the PTPRO protein into contact with the candidate cells.

In some embodiments, the method comprises bringing primers capable of specifically amplifying a nucleic acid molecule encoding the PTPRO and/or probes capable of specifically recognizing the nucleic acid molecule encoding the PTPRO into contact with the candidate cells.

In some embodiments, the method comprises determining whether the candidate cells have the following feature: NTRK3⁺.

In some embodiments, the determining comprises directly or indirectly detecting the candidate cells for expression and/or activity level of NTRK3.

In some embodiments, the expression and/or activity level of NTRK3 comprises expression and/or activity level of a nucleic acid molecule encoding the NTRK3, and/or expression and/or activity level of an NTRK3 protein.

In some embodiments, the detecting comprises modifying the candidate cells.

In some embodiments, the detecting comprises use of marker molecules.

In some embodiments, the marker molecules comprise proteins, nucleic acids and/or small molecules.

In some embodiments, the marker molecules comprise fluorescent reporter genes.

In some embodiments, the method comprises bringing a reagent capable of specifically binding to the NTRK3 protein and/or a reagent capable of measuring activity of the NTRK3 protein into contact with the candidate cells.

In some embodiments, the method comprises bringing primers capable of specifically amplifying a nucleic acid molecule encoding the NTRK3 and/or probes capable of specifically recognizing the nucleic acid molecule encoding the NTRK3 into contact with the candidate cells.

In some embodiments, the method comprises determining whether the candidate cells have the following feature: FLRT2⁺.

In some embodiments, the determining comprises directly or indirectly detecting the candidate cells for expression and/or activity level of FLRT2.

In some embodiments, the expression and/or activity level of FLRT2 comprises expression and/or activity level of a nucleic acid molecule encoding the FLRT2, and/or expression and/or activity level of an FLRT2 protein.

In some embodiments, the detecting comprises modifying the candidate cells.

In some embodiments, the detecting comprises use of marker molecules.

In some embodiments, the marker molecules comprise proteins, nucleic acids and/or small molecules.

In some embodiments, the marker molecules comprise fluorescent reporter genes.

In some embodiments, the method comprises bringing a reagent capable of specifically binding to the FLRT2 protein and/or a reagent capable of measuring activity of the FLRT2 protein into contact with the candidate cells.

In some embodiments, the method comprises bringing primers capable of specifically amplifying a nucleic acid molecule encoding the FLRT2 and/or probes capable of specifically recognizing the nucleic acid molecule encoding the FLRT2 into contact with the candidate cells.

In some embodiments, the method comprises determining whether the candidate cells have the following feature: KITLG⁺.

In some embodiments, the determining comprises directly or indirectly detecting the candidate cells for expression and/or activity level of KITLG.

In some embodiments, the expression and/or activity level of KITLG comprises expression and/or activity level of a nucleic acid molecule encoding the KITLG, and/or expression and/or activity level of a KITLG protein.

In some embodiments, the detecting comprises modifying the candidate cells.

In some embodiments, the detecting comprises use of marker molecules.

In some embodiments, the marker molecules comprise proteins, nucleic acids and/or small molecules.

In some embodiments, the marker molecules comprise fluorescent reporter genes.

In some embodiments, the method comprises bringing a reagent capable of specifically binding to the KITLG protein and/or a reagent capable of measuring activity of the KITLG protein into contact with the candidate cells.

In some embodiments, the method comprises bringing primers capable of specifically amplifying a nucleic acid molecule encoding the KITLG and/or probes capable of specifically recognizing the nucleic acid molecule encoding the KITLG into contact with the candidate cells.

In some embodiments, the method comprises determining whether the candidate cells have the following feature: CD83⁺.

In some embodiments, the determining comprises directly or indirectly detecting the candidate cells for expression and/or activity level of CD83.

In some embodiments, the expression and/or activity level of CD83 comprises expression and/or activity level of a nucleic acid molecule encoding the CD83, and/or expression and/or activity level of a CD83 protein.

In some embodiments, the detecting comprises modifying the candidate cells.

In some embodiments, the detecting comprises use of marker molecules.

In some embodiments, the marker molecules comprise proteins, nucleic acids and/or small molecules.

In some embodiments, the marker molecules comprise fluorescent reporter genes.

In some embodiments, the method comprises bringing a reagent capable of specifically binding to the CD83 protein and/or a reagent capable of measuring activity of the CD83 protein into contact with the candidate cells.

In some embodiments, the method comprises bringing primers capable of specifically amplifying a nucleic acid molecule encoding the CD83 and/or probes capable of specifically recognizing the nucleic acid molecule encoding the CD83 into contact with the candidate cells.

In some embodiments, the method comprises determining whether the candidate cells have the following feature: LMX1A+EN1⁺.

In some embodiments, the determining comprises directly or indirectly detecting the candidate cells for expression and/or activity level of LMX1A, and for expression and/or activity level of EN1.

In some embodiments, the expression and/or activity level of LMX1A comprises expression and/or activity level of a nucleic acid molecule encoding the LMX1A, and/or expression and/or activity level of a LMX1A protein.

In some embodiments, the expression and/or activity level of EN1 comprises expression and/or activity level of a nucleic acid molecule encoding the EN1, and/or expression and/or activity level of an EN1 protein.

In some embodiments, the detecting comprises modifying the candidate cells.

In some embodiments, the detecting comprises use of marker molecules.

In some embodiments, the marker molecules comprise proteins, nucleic acids and/or small molecules.

In some embodiments, the marker molecules comprise fluorescent reporter genes.

In some embodiments, the method comprises bringing a reagent capable of specifically binding to the LMX1A protein and/or a reagent capable of measuring activity of the LMX1A protein into contact with the candidate cells.

In some embodiments, the method comprises bringing primers capable of specifically amplifying a nucleic acid molecule encoding the LMX1A and/or probes capable of specifically recognizing the nucleic acid molecule encoding the LMX1A into contact with the candidate cells.

In some embodiments, the method comprises bringing a reagent capable of specifically binding to the EN1 protein and/or a reagent capable of measuring activity of the EN1 protein into contact with the candidate cells.

In some embodiments, the method comprises bringing primers capable of specifically amplifying a nucleic acid molecule encoding the EN1 and/or probes capable of specifically recognizing the nucleic acid molecule encoding the EN1 into contact with the candidate cells.

In another aspect, the present application provides a cell product, comprising dopaminergic neural precursor cells obtained with the method defined.

In another aspect, the present application provides a method for isolating dopaminergic neural precursor cells, comprising: (a) providing a neural precursor cell population, and (b) isolating, from the neural precursor cell population, cells having one or more of the following features: CLSTN2⁺, PTPRO⁺, NTRK3⁺, FLRT2⁺, KITLG⁺, CD83⁺, and/or LMX1A⁺EN1⁺.

In another aspect, the present application provides a method for enriching dopaminergic neural precursor cells, comprising: (a) providing a neural precursor cell population, and (b) enriching, from the neural precursor cell population, cells having one or more of the following features: CLSTN2⁺, PTPRO⁺, NTRK3⁺, FLRT2⁺, KITLG⁺, CD83⁺, and/or LMX1A⁺EN1⁺.

In some embodiments, the method comprises: (a) providing a neural precursor cell population, and (b) isolating or enriching cells having the CLSTN2⁺ from the neural precursor cell population.

In some embodiments, the method comprises: (a) providing a neural precursor cell population, and (b) isolating or enriching cells having the CLSTN2⁺ from the neural precursor cell population.

In some embodiments, the method comprises: (a) providing a neural precursor cell population, and (b) isolating or enriching cells having the LMX1A⁺EN1⁺ from the neural precursor cell population.

In another aspect, the present application provides a dopaminergic neural precursor cell population, comprising dopaminergic neural precursor cells obtained according to the method as defined.

In another aspect, the present application provides a method for preparing a cell product, comprising: (a) providing neural precursor cells, and (b) isolating and/or enriching neural precursor cells having one or more of the following features: CLSTN2⁺, PTPRO⁺, NTRK3⁺, FLRT2⁺, KITLG⁺, CD83⁺, and/or LMX1A+EN1⁺.

In some embodiments, the method comprises differentiating a cell population to obtain the neural precursor cells.

In some embodiments, the cell population is derived from rodent cells, primate cells, or human cells.

In some embodiments, the cell population is derived from pluripotent stem cells.

In some embodiments, the cell population is derived from human pluripotent stem cells.

In some embodiments, the method comprises contacting the cell population with an ALK inhibitor, a sonic hedgehog (SHH) signaling activator and a GSK-3 inhibitor.

In some embodiments, the ALK inhibitor comprises an ALK2 inhibitor, an ALK4 inhibitor, an ALK5 inhibitor, and/or an ALK7 inhibitor.

In some embodiments, the ALK4 inhibitor comprises SB431542.

In some embodiments, the ALK2 inhibitor comprises DMH-1.

In some embodiments, the SHH signaling activator comprises SHH C25II, SAG, and/or Purmorphamine.

In some embodiments, the GSK-3 inhibitor comprises CHIR99021.

In some embodiments, the contacting is performed under a condition enabling differentiation of the cell population into midbrain floor plate precursor cells.

In some embodiments, the neural precursor cells can be differentiated into neural cells comprising at least 30% of dopaminergic neural cells.

In some embodiments, the differentiation comprises in vitro differentiation and in vivo differentiation.

In another aspect, the present application provides a method for evaluating a cell product, comprising: performing evaluation by detecting the cell product for a proportion of cells having one or more of the following features: CLSTN2⁺, PTPRO⁺, NTRK3⁺, FLRT2⁺, KITLG⁺, CD83⁺, and/or LMX1A⁺EN1⁺.

In another aspect, the present application provides a method for optimizing a preparation process for a cell product, comprising: performing optimization by detecting the cell product for a proportion of cells having one or more of the following features: CLSTN2⁺, PTPRO⁺, NTRK3⁺, FLRT2⁺, KITLG⁺, CD83⁺, and/or LMX1A+EN1⁺.

In some embodiments, the preparation process comprises optimizing generation, differentiation, isolation, and/or purification of the cell product.

In another aspect, the present application provides a cell preparation obtained by further amplification and proliferation of the cell product.

In another aspect, the present application provides use of a CLSTN2⁺ indicator in preparation of a cell product, wherein the cell product comprises dopaminergic neural precursor cells.

In another aspect, the present application provides use of a PTPRO⁺ indicator in preparation of a cell product, wherein the cell product comprises dopaminergic neural precursor cells.

In another aspect, the present application provides use of an NTRK3⁺ indicator in preparation of a cell product, wherein the cell product comprises dopaminergic neural precursor cells.

In another aspect, the present application provides use of an FLRT2⁺ indicator in preparation of a cell product, wherein the cell product comprises dopaminergic neural precursor cells.

In another aspect, the present application provides use of a KITLG⁺ indicator in preparation of a cell product, wherein the cell product comprises dopaminergic neural precursor cells.

In another aspect, the present application provides use of a CD83⁺ indicator in preparation of a cell product, wherein the cell product comprises dopaminergic neural precursor cells.

In another aspect, the present application provides use of an LMX1A⁺EN1⁺ indicator in preparation of a cell product, wherein the cell product comprises dopaminergic neural precursor cells.

In another aspect, the present application provides a quality control kit for preparing dopaminergic neural precursor cells, comprising a quality control reagent capable of determining whether candidate cells have one or more of the following features: CLSTN2⁺, PTPRO⁺, NTRK3⁺, FLRT2⁺, KITLG⁺, CD83⁺, and/or LMX1A⁺EN1⁺.

In some embodiments, the kit comprises a reagent capable of culturing and/or preserving the candidate cells.

In some embodiments, the candidate cells are neural precursor cells.

In some embodiments, the candidate cells are derived from pluripotent stem cells.

In some embodiments, the candidate cells are derived from human pluripotent stem cells.

In some embodiments, the candidate cells have differentiated in vitro for at least about 10 days.

In some embodiments, the reagent capable of culturing and/or preserving the candidate cells is packaged independently with the quality control reagent.

In some embodiments, the quality control reagent is capable of directly or indirectly detecting the candidate cells for expression and/or activity level of CLSTN2.

In some embodiments, the expression and/or activity level of CLSTN2 comprises expression and/or activity level of a nucleic acid molecule encoding the CLSTN2, and/or expression and/or activity level of an CLSTN2 protein.

In some embodiments, the quality control reagent comprises primers capable of specifically amplifying a nucleic acid molecule encoding the CLSTN2 and/or probes capable of specifically recognizing the nucleic acid molecule encoding the CLSTN2.

In some embodiments, the quality control reagent is capable of directly or indirectly detecting the candidate cells for expression and/or activity level of PTPRO.

In some embodiments, the expression and/or activity level of PTPRO comprises expression and/or activity level of a nucleic acid molecule encoding the PTPRO, and/or expression and/or activity level of an PTPRO protein.

In some embodiments, the quality control reagent comprises primers capable of specifically amplifying a nucleic acid molecule encoding the PTPRO and/or probes capable of specifically recognizing the nucleic acid molecule encoding the PTPRO.

In some embodiments, the quality control reagent is capable of directly or indirectly detecting the candidate cells for expression and/or activity level of NTRK3.

In some embodiments, the expression and/or activity level of NTRK3 comprises expression and/or activity level of a nucleic acid molecule encoding the NTRK3, and/or expression and/or activity level of an NTRK3 protein.

In some embodiments, the quality control reagent comprises primers capable of specifically amplifying a nucleic acid molecule encoding the NTRK3 and/or probes capable of specifically recognizing the nucleic acid molecule encoding the NTRK3.

In some embodiments, the quality control reagent is capable of directly or indirectly detecting the candidate cells for expression and/or activity level of FLRT2.

In some embodiments, the expression and/or activity level of FLRT2 comprises expression and/or activity level of a nucleic acid molecule encoding the FLRT2, and/or expression and/or activity level of an FLRT2 protein.

In some embodiments, the quality control reagent comprises primers capable of specifically amplifying a nucleic acid molecule encoding the FLRT2 and/or probes capable of specifically recognizing the nucleic acid molecule encoding the FLRT2.

In some embodiments, the quality control reagent is capable of directly or indirectly detecting the candidate cells for expression and/or activity level of KITLG.

In some embodiments, the expression and/or activity level of KITLG comprises expression and/or activity level of a nucleic acid molecule encoding the KITLG, and/or expression and/or activity level of an KITLG protein.

In some embodiments, the quality control reagent comprises primers capable of specifically amplifying a nucleic acid molecule encoding the KITLG and/or probes capable of specifically recognizing the nucleic acid molecule encoding the KITLG.

In some embodiments, the quality control reagent is capable of directly or indirectly detecting the candidate cells for expression and/or activity level of CD83.

In some embodiments, the expression and/or activity level of CD83 comprises expression and/or activity level of a nucleic acid molecule encoding the CD83, and/or expression and/or activity level of a CD83 protein.

In some embodiments, the quality control reagent comprises primers capable of specifically amplifying a nucleic acid molecule encoding the CD83 and/or probes capable of specifically recognizing the nucleic acid molecule encoding the CD83.

In some embodiments, the quality control reagent is capable of directly or indirectly detecting the candidate cells for expression and/or activity level of LMX1A, and for expression and/or activity level of EN1.

In some embodiments, the expression and/or activity level of LMX1A comprises expression and/or activity level of a nucleic acid molecule encoding the LMX1A, and/or expression and/or activity level of a LMX1A protein.

In some embodiments, the expression and/or activity level of EN1 comprises expression and/or activity level of a nucleic acid molecule encoding the EN1, and/or expression and/or activity level of an EN1 protein.

In some embodiments, the quality control reagent comprises a reagent capable of specifically binding to the LMX1A protein and/or a reagent capable of measuring activity of the LMX1A protein.

In some embodiments, the quality control reagent comprises a reagent capable of specifically binding to the EN1 protein and/or a reagent capable of measuring activity of the EN1 protein.

In another aspect, the present application provides a method for controlling quality of prepared dopaminergic neural precursor cells, comprising the following steps: a) detecting the prepared cells for a proportion of cells having one or more of the following features: CLSTN2⁺, PTPRO⁺, NTRK3⁺, FLRT2⁺, KITLG⁺, CD83⁺, and/or LMX1A⁺EN1⁺; and b) if the proportion detected in step a) is at least about 10%, determining that the quality of the prepared dopaminergic neural precursor cells is acceptable.

In some embodiments, if the proportion detected in step a) is at least about 30%, the quality of the prepared dopaminergic neural precursor cells is acceptable.

In another aspect, the present application provides an isolated or enriched dopaminergic neural precursor cell population, which expresses one or more of the following dopaminergic neural precursor cell markers: CLSTN2⁺, PTPRO⁺, NTRK3⁺, FLRT2⁺, KITLG⁺, CD83⁺, and/or LMX1A⁺EN1⁺.

In some embodiments, the isolated or enriched dopaminergic neural precursor cell population has a feature of CLSTN2⁺.

In some embodiments, the isolated or enriched dopaminergic neural precursor cell population has a feature of PTPRO⁺.

In some embodiments, the isolated or enriched dopaminergic neural precursor cell population has a feature of NTRK3⁺.

In some embodiments, the isolated or enriched dopaminergic neural precursor cell population has a feature of FLRT2⁺.

In some embodiments, the isolated or enriched dopaminergic neural precursor cell population has a feature of KITLG⁺.

In some embodiments, the isolated or enriched dopaminergic neural precursor cell population has a feature of CD83⁺.

In some embodiments, the isolated or enriched dopaminergic neural precursor cell population has a feature of LMX1A⁺EN1⁺.

In another aspect, the present application provides a dopaminergic neural precursor cell population, wherein at least about 10%, 20%, 30%, 40%, 50%, 60%, 70%, 80%, or 90% of cells express one or more of the following dopaminergic neural precursor cell markers: CLSTN2⁺, PTPRO⁺, NTRK3⁺, FLRT2⁺, KITLG⁺, CD83⁺, and/or LMX1A⁺EN1⁺.

In some embodiments, in the dopaminergic neural precursor cell population, at least about 10%, 20%, 30%, 40%, 50%, 60%, 70%, 80%, or 90% of cells express CLSTN2.

In some embodiments, in the dopaminergic neural precursor cell population, at least about 10%, 20%, 30%, 40%, 50%, 60%, 70%, 80%, or 90% of cells express PTPRO.

In some embodiments, in the dopaminergic neural precursor cell population, at least about 10%, 20%, 30%, 40%, 50%, 60%, 70%, 80%, or 90% of cells express NTRK3.

In some embodiments, in the dopaminergic neural precursor cell population, at least about 10%, 20%, 30%, 40%, 50%, 60%, 70%, 80%, or 90% of cells express FLRT2.

In some embodiments, in the dopaminergic neural precursor cell population, at least about 10%, 20%, 30%, 40%, 50%, 60%, 70%, 80%, or 90% of cells express KITLG.

In some embodiments, in the dopaminergic neural precursor cell population, at least about 10%, 20%, 30%, 40%, 50%, 60%, 70%, 80%, or 90% of cells express CD83.

In some embodiments, in the dopaminergic neural precursor cell population, at least about 10%, 20%, 30%, 40%, 50%, 60%, 70%, 80%, or 90% of cells express LMX1A and EN1.

In some embodiments, in the dopaminergic neural precursor cell population, at least about 40%, 50%, 60%, 70%, 80%, or 90% of cells express one or more of the following dopaminergic neural precursor cell markers: CLSTN2⁺, PTPRO⁺, NTRK3⁺, FLRT2⁺, KITLG⁺, CD83⁺, and/or LMX1A⁺EN1⁺.

In some embodiments, in the dopaminergic neural precursor cell population, at least about 40%, 50%, 60%, 70%, 80%, or 90% of cells express CLSTN2.

In some embodiments, in the dopaminergic neural precursor cell population, at least about 40%, 50%, 60%, 70%, 80%, or 90% of cells express PTPRO.

In some embodiments, in the dopaminergic neural precursor cell population, at least about 40%, 50%, 60%, 70%, 80%, or 90% of cells express NTRK3.

In some embodiments, in the dopaminergic neural precursor cell population, at least about 40%, 50%, 60%, 70%, 80%, or 90% of cells express PTPRO⁺.

In some embodiments, in the dopaminergic neural precursor cell population, at least about 40%, 50%, 60%, 70%, 80%, or 90% of cells express FLRT2.

In some embodiments, in the dopaminergic neural precursor cell population, at least about 40%, 50%, 60%, 70%, 80%, or 90% of cells express CD83.

In some embodiments, in the dopaminergic neural precursor cell population, at least about 40%, 50%, 60%, 70%, 80%, or 90% of cells express LMX1A and EN1.

In another aspect, the present application provides a cell product, comprising dopaminergic neural precursor cells obtained according to the method of the present application.

In another aspect, the present application provides a cell product, comprising the dopaminergic neural precursor cell population of the present application.

In another aspect, the present application provides a graft composition prepared from in vivo or in vitro differentiation of the dopaminergic neural precursor cell population of the present application.

In another aspect, the present application provides a pharmaceutical composition, comprising the dopaminergic neural precursor cell population of the present application or the cell product of the present application.

In some embodiments, the pharmaceutical composition further comprises a pharmaceutically acceptable adjuvant.

In another aspect, the present application provides a method for preventing, treating, or alleviating nervous system diseases or disorders, comprising administering the dopaminergic neural precursor cell population, the cell product, and/or the pharmaceutical composition as defined in the present application to a subject in need thereof.

In some embodiments, the neurological diseases or disorders comprise Parkinson's disease.

In another aspect, the present application provides use of the dopaminergic neural precursor cell population, the cell product, and/or the pharmaceutical composition as defined in the present application in preparation of medicaments for prevention, treatment or alleviation of nervous system diseases or disorders.

In some embodiments, the neurological diseases or disorders comprise Parkinson's disease.

In another aspect, the present application provides use of the dopaminergic neural precursor cell population, the cell product, and/or the pharmaceutical composition as defined in prevention, treatment or alleviation of nervous system diseases or disorders.

In some embodiments, the neurological diseases or disorders comprise Parkinson's disease.

In another aspect, the present application further provides grafts (for example, graft compositions) after in vivo or in vitro differentiation, and use of these grafts or graft compositions.

Other aspects and advantages of the present application may be readily perceived by those skilled in the art from the detailed description below. The detailed description below only illustrates and describes the exemplary embodiments of the present application. As would be appreciated by those skilled in the art, the content of the present application allows those killed in the art to change the specific embodiments disclosed without departing from the spirit and scope involved in the present application. Accordingly, the accompanying drawings and the description in the specification of the present application are merely for an exemplary but not restrictive purpose.

### BRIEF DESCRIPTION OF THE DRAWINGS

The specific features of the invention involved in the present application are listed in the appended claims. The characteristics and advantages of the invention involved in the present application may be better understood by referring to the exemplary embodiments and the accompanying drawings described in detail below. The accompanying drawings are briefly illustrated as follows.

FIG. 1 shows a schematic diagram of sampling time points for in vitro differentiation of human stem cells undergoing scRNA-seq.

FIG. 2 shows the clustering results for each stage of in vitro mDA differentiation of human stem cells visualized using t-SNE. The horizontal ratio bar indicates the proportions of cell types and is displayed with the corresponding shades of cell populations.

FIG. 3 and FIG. 4 show the expression of multiple key transcription factors (for example, EN1 and LMX1A) of the mDA lineage and the midbrain ventral floor plate neural precursor cell markers in the umap clustering plots of the precursor cell population on Days 21 (FIG. 3) and 28 (FIG. 4).

FIG. 5 shows the expression of two key transcription factors (EN1 and LMX1A) of the mDA lineage as well as CLSTN2 and PTPRO in the umap clustering plots of the precursor cell population on Days 21 (top) and 28 (bottom).

FIG. 6 shows the expression of NTRK3, FLRT2, KITLG, and CD83 in umap clustering with scRNA-seq data on Day 21 (left) and scRNA-seq data on Day 28 (right).

FIG. 7 shows a schematic diagram of a double reporter cell line and the joint batch analysis of RNA-seq and scRNA-seq data.

FIG. 8 shows PCR identification of an LMX1A-tdTomato hESC line (left panel of FIG. 8), where expected PCR products of LMX1A gene editing based on the correctly targeted left arm (LA) and right arm (RA) of a recombinant arm are ˜1500 bp and ˜3,000 bp, respectively. Homozygous clones are identified by PCR products of ˜1,000 bp. Those clones without PCR products of ˜1,000 bp are homozygous clones. A mother cell line (H9 ESC) is used as a control, and PCR identification was performed on the LMX1A-tdTomato/EN1-mNeonGreen hESC line (right panel in FIG. 8). The expected PCR products of EN1 gene editing based on correctly targeted left arm (LA) and right arm (RA) of the recombinant arm are ˜1700 bp and ˜3300 bp, respectively. Homozygous clones are identified by PCR products of ˜700 bp. Those clones without PCR products of ˜700 bp were homozygous clones. The mother cell line (H9 ESC) was used as a control. The clones selected were heterozygous.

FIG. 9 shows a typical FACS plot of mDA differentiation using the LMX1A-tdTomato/EN1-mNeonGreen hESC line on Day 21. LMX1A⁺EN1⁺ cells account for about 30% of the total cells.

FIG. 10 shows the DEG expression of four groups of cell populations (LMX1A⁺EN1⁺ on Day 21, Others on Day 21, LMX1A⁺EN1⁺ on Day 28 and Others on Day 28) in a heat map. Others represent other cells that are collected together with LMX1A⁺EN1⁻, LMX1A⁺EN1⁻ and LMX1A− EN1⁺. Representative marker genes are listed on the right side of the heat map. The genes in the box are from LMX1A⁺EN1⁺ DEGs on Day 21 and LMX1A⁺ EN1⁺ DEGs on Day 28.

FIG. 11 shows the differentially expressed genes in LMX1A⁺ EN1⁺ and other cells on Day 21, LMX1A⁺ EN1⁺ and other cells on Day 28, as well as LMX1A⁺EN1⁺ cells on Day 21 and LMX1A⁺EN1⁺ cells on Day 28, in volcano maps. The assigned DEGs (points within the box) were determined using the threshold: Log 2 fold change >2, and p-value=10-3. The enlarged dots were marker genes (LMX1A, HOXA2, PHOX2B, and OLIG2) representing cell types, or molecular marker genes (CLST2N, PTPRO, and NTRK3). Compared with the LMX1A⁺EN1⁺ group on Day 28, the enlarged points (PTPRO, the region between two boxes in each map) of the LMX1A⁺EN1⁺ on Day 21 indicate that PTPRO is significant, but does not reach the Log 2 fold change threshold.

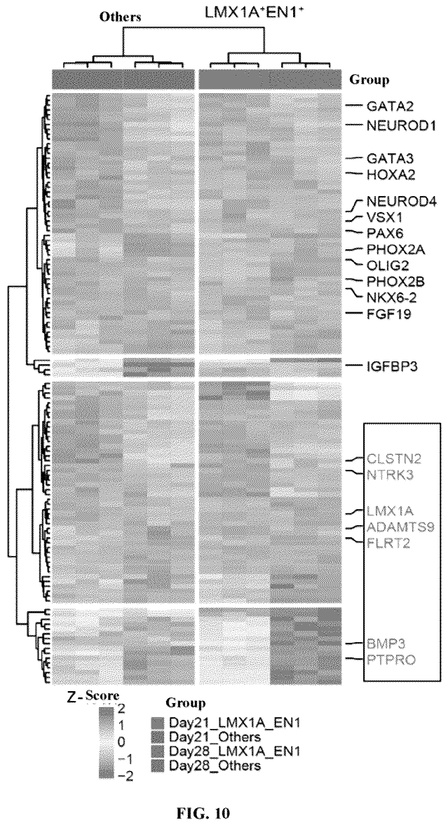

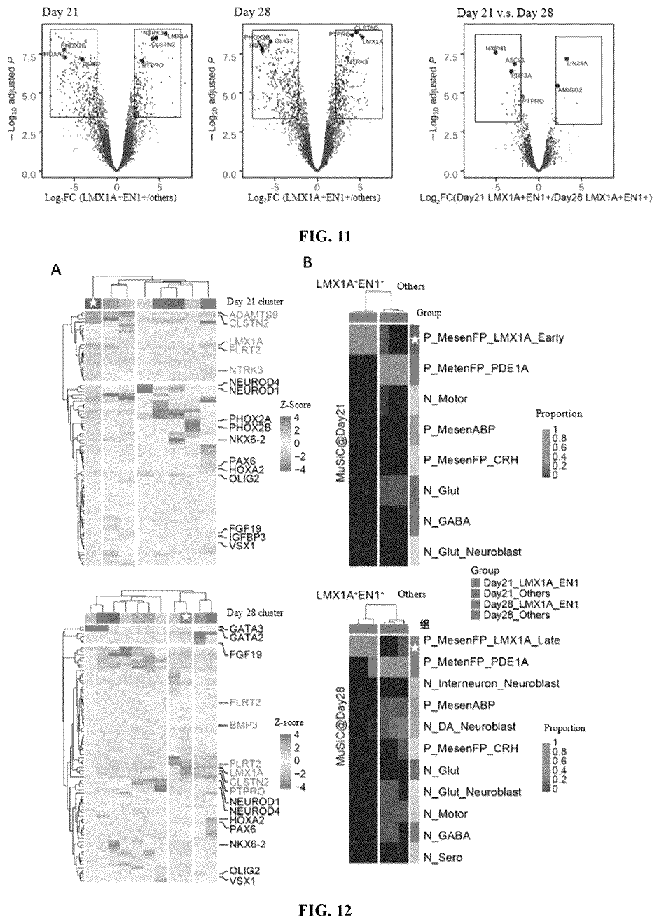

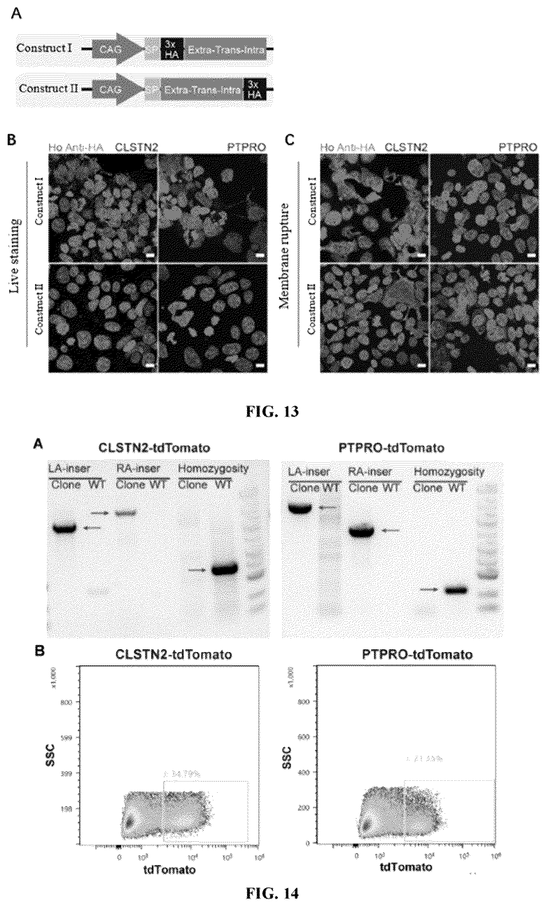

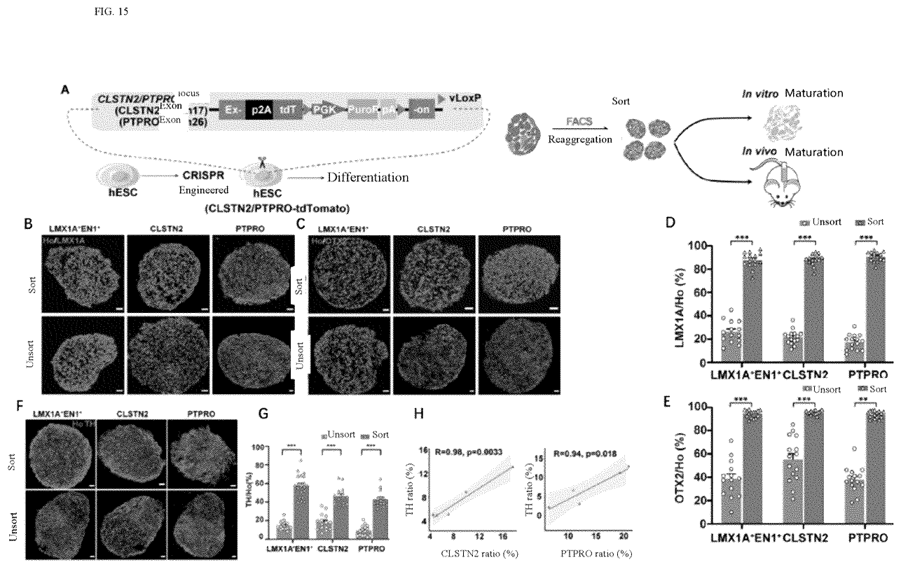

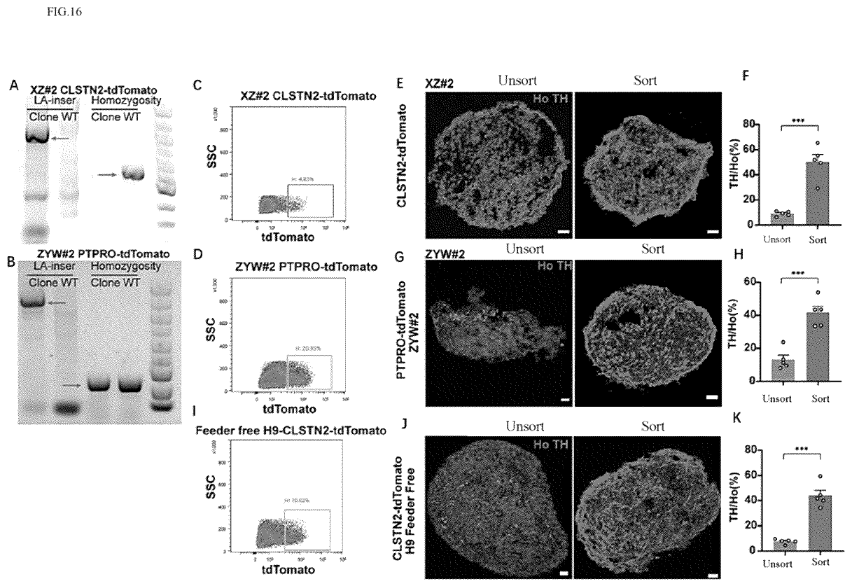

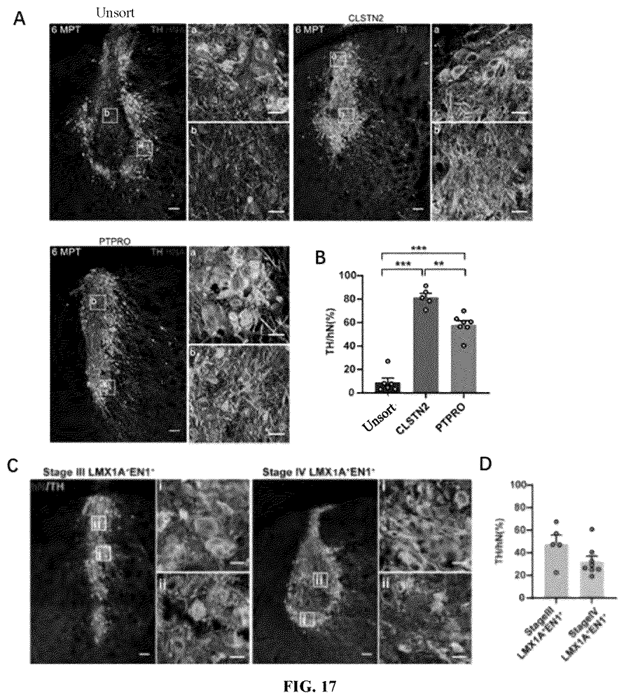

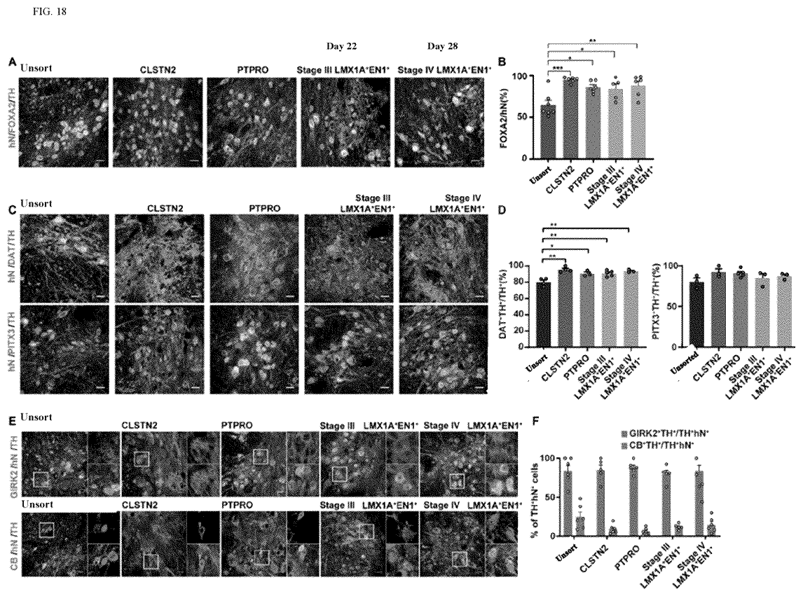

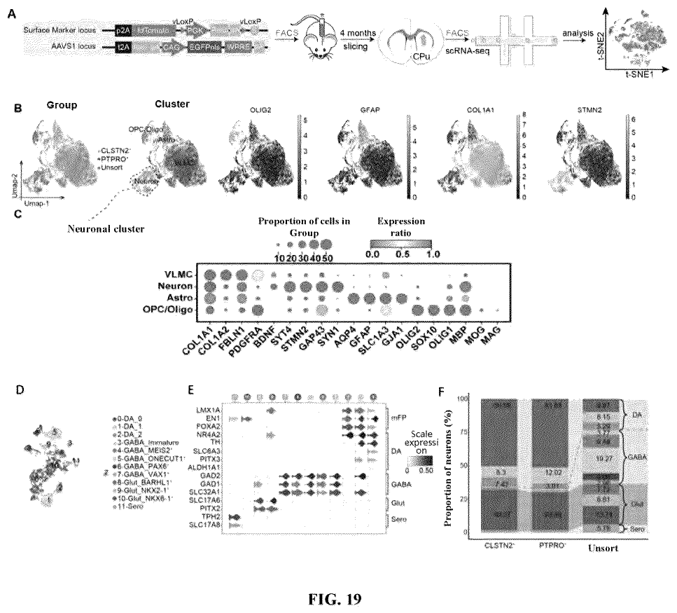

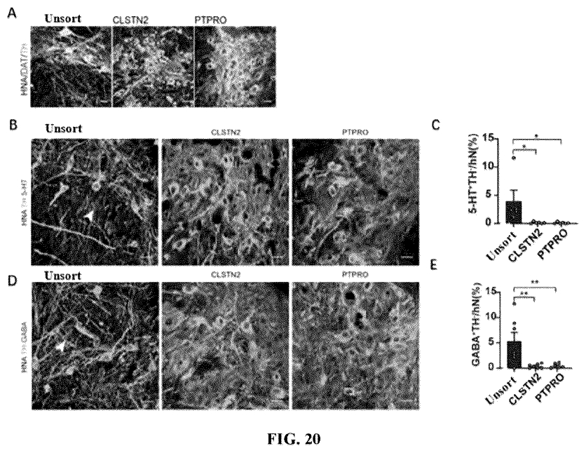

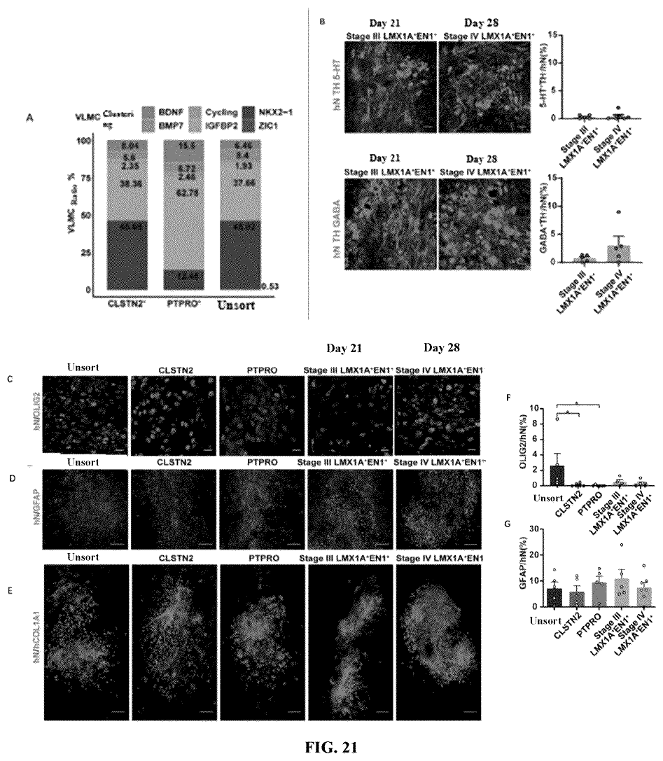

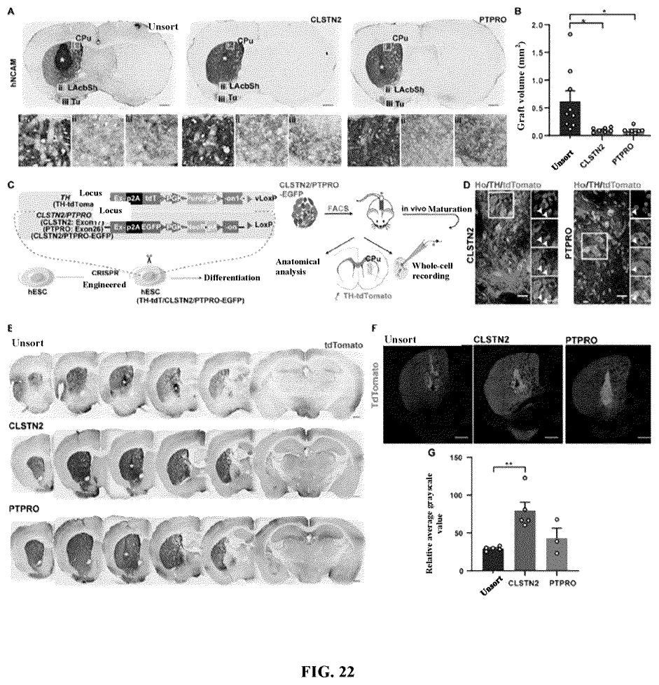

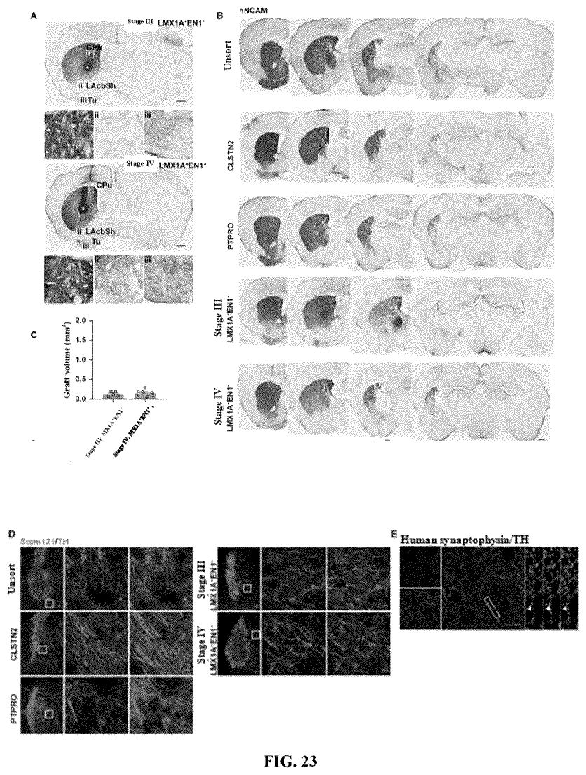

In FIG. 12, A shows the expression of cell populations on Days 21 (top) and 28 (bottom), respectively, using the same DEG in FIG. 10. The populations with the asterisks are P_MesenFP_LMX1A_Early on Day 21 and P_MesenFP_LMX1A_Late on Day 28, both of which are assumed mDA precursor cell populations. The genes in the boxes are from the LMX1A⁺EN1⁺ DEGs on Day 21 and of the LMX1A⁺EN1⁺ DEGs on Day 28, as in FIG. 10. In FIG. 12, B shows a heat map in which the cell type proportion estimation is performed on the cell populations of the LMX1A⁺EN1⁺ and Others in batch RNA sequenced samples, based on the scRNA-seq data on Days 21 (top) and 28 (bottom), respectively. Among the included cell types, the estimated proportions were normalized to a sum of 1. The populations with asterisks are P_MesenFP_LMX1A_Early on Day 21 and P_MesenFP_LMX1A_Late on Day 28, both of which are assumed mDA precursor cell populations.

In FIG. 13, A shows schematic diagrams of two plasmid constructs. SP represents the signaling peptide of a selected surface marker gene. In Construct I, a 3×HA tag was inserted between the SP and the rest of the amino acid coding sequences, while in Construct II, the 3×HA tag was fused after the amino acid sequence of the surface marker gene. In B and C of FIG. 13, in HEK293T cells, calcium phosphate transfection constructs I (top) and II (bottom) were treated immunocytochemically with an anti-HA antibody for live staining (B) and immobilization/membrane rupture (C), respectively. For live-stained cell immunity, HA signaling is only detectable in Construct I where the 3×HA tag was located outside the cell membrane, but undetectable in Construct II where the 3×HA was located in the intracellular domain (B).

FIG. 14 shows the construction of surface molecular marker reporter cell lines. FIG. 14A shows the PCR genotyping for CLSTN2- and PTPRO-tdT hPSC cell lines. The PCR products expected to be inserted in CLSTN2 gene loci for LA and RA were ˜2200 bp and ˜2800 bp, respectively. The homozygote was ˜680 bp. The PCR products expected to be inserted in PTPRO gene loci for LA and RA were ˜3,000 bp and ˜1600 bp, respectively; and the homozygote is ˜300 bp. FIG. 14B shows typical FACS images of mDA differentiation in Stage III (CLSTN2) and Stage IV (PTPRO), using CSLTN2- or PTRPO-tdT hPSC cell lines.

FIG. 15 shows the in vitro enrichment of mDA neurons by a molecular marker combination of LMX1A and EN1, by CLSTN2 and by PTPRO. Here, FIG. 15A shows a schematic diagram of gene editing for a marker gene reporter cell line and a schematic experimental diagram of in vitro and in vivo maturation of sorted neural cells. The precursor cells were sorted by FACS and re-aggregated into neurospheres, which were then matured in vitro or transplanted into the brains of PD mice for in vivo maturation. FIGS. 15B-15E show neurospheres immunostained in vitro with antibodies to the ventral midbrain marker molecules LMX1A (FIG. 15B) and OTX2 (FIG. 15C). The scale bar corresponds to 25 μm. The quantitative ratios of LMX1A+ cells (FIG. 15D) and OTX2+ cells (FIG. 15E) in neurospheres are shown. The multiple unpaired t-test was corrected using Holm-Sidak. FIGS. 15F-15H show that CLSTN2 and PTPRO can predict mDA neuronal differentiation, and generate highly enriched mDA neurons after the neural precursor cells are sorted and matured in vitro. FIG. 15F shows the results of TH+. FIG. 15G shows the results of the multiple unpaired t-test corrected using Holm-Sidak, in 3 batches, with 5 neurospheres per batch. The scale bar corresponds to 25 μm. FIG. 15H shows the correlation between the ratio of CLSTN2-expressing neural precursor cells and the ratio of TH neurons within sorted and enriched mature neurospheres (left: CLSTN2-TH; right: PTPRO-TH).

FIG. 16 shows the construction of hiPSCs surface molecular marker reporter cell lines, the effect of surface molecular markers on hiPSC-derived mDA neural cells, and the enrichment of mDA neural cells under feeder-free conditions. CLSTN2 or PTPRO reporter cell lines were constructed on two separate hiPSC cell lines. FIGS. 16A and 16B show the PCR genotyping for CLSTN2- and PTPRO-tdT hiPSC cell lines, respectively, with the corresponding mother cell line (XZ #2 hiPSC line or ZYW #2 hiPSC, labeled as WT) as a control. FIGS. 16C-16D show the typical images of FACS assay for HIPSC-derived neural precursor cells, using CSLTN2-tdT (FIG. 16C) hiPSCs in Stage III (CLSTN2) or using PTPRO-tdT (FIG. 16D) hiPSC cell lines in Stage IV (PTPRO). FIGS. 16E-16H show typical plots of in vitro TH immunostaining of in vitro mature neurospheres in the sorting and non-sorting groups, and the statistical results of the ratio of TH-positive cell. FIG. 16I shows the ratio of CLSTN2-positive cells in neural precursors derived from hESCs under feeder-free conditions. FIGS. 16J-16K show the in vitro enrichment effect of CLSTN2 on hESC-derived mDA neurons under feeder-free conditions. The scale bar corresponds to 25 μm. Non-paired t-test was conducted. Data were expressed as mean±SEM. *** p<0.001.

FIG. 17 shows the in vivo enrichment of mDA neurons by a molecular marker combination of LMX1A and EN1, by CLSTN2 and by PTPRO. Here, FIG. 17A shows grafts, which are derived from unsorted precursor cells, CLSTN2-sorted precursor cells and PTPRO-sorted precursor cells, respectively, and immunostained with human nuclei (HNA) and TH. FIG. 17B shows quantification results of the ratio of TH+ neurons in the grafts. N=6 (unsorted), 5 (CLSTN2), and 7 (PTPRO). One-way analysis of variance was followed by Tukey's multiple comparison test. FIG. 17C shows the results of human nuclei (hN) and TH immunostaining of grafts derived from LMX1A⁺EN1⁺ neural precursor cells in Stage III and LMX1A⁺EN1⁺ neural precursor cells in Stage IV. The scale bar corresponds to 100 μm. Here, (i) and (ii) represent the marginal and central regions of the grafts, respectively. The scale bar corresponds to 20 μm. FIG. 17D shows quantification of TH+ neuron ratios in grafts. N=5 (LMX1A⁺EN1⁺ in Stage III) and 8 (LMX1A⁺EN1⁺ in Stage IV). One-way analysis of variance was followed by Tukey's multiple comparison test.

FIG. 18 shows the cellular properties of enriching mDA neurons. The grafts were subjected to typical immunostaining with FOXA2, hN and TH (FIG. 18A) and FOXA2+ cells (FIG. 18B) at quantified ratios. The scale bar corresponds to 20 μm. N=7 (unsorted), 6 (CLSTN2), 6 (PTPRO), 5 (LMX1A⁺EN1⁺ in Stage III), and 6 (LMX1A⁺EN1⁺ in Stage IV). One-way analysis of variance was followed by Tukey's multiple comparison test. * p<0.05, ** p<0.01, and *** p<0.001. FIG. 18C shows graft immunostaining with hN and DAT (top) or PITX3 (bottom). The scale bar corresponds to 20 μm. FIG. 18D: the quantification of the ratios of DAT⁺TH⁺/TH⁺ cells (left) and PITX3⁺TH⁺/TH⁺ cells (right) are shown. FIGS. 18E and 18F: the grafts immunostained with hN and GIRK2 (top) or CB (bottom) and the quantification of the ratio of mDA isotypes in total TH+ neuronal ratios (F) are shown. The scale bar corresponds to 20 μm. Data were expressed as mean±SEM. * p<0.05, ** p<0.01, and *** p<0.001.

FIG. 19 shows the analysis of cell composition after transplantation of mDA precursor cells enriched by transplantation sorting or non-sorting. Here, (A) shows a schematic diagram and schematic experimental diagram of the cell line for single-cell sequencing of grafts; (B) represents the typical gene expression of major cell types of the grafts; (C) represents the composition of major cell types in the grafts; (D) shows further clustering analysis of neural cells: (E) shows typical marker genes of each type of neural cells: and (F) shows the ratio of neural cell subtypes per group of grafts (the non-sorting group, the CLSTN2 sorting group, and the PTPRO sorting group).

In FIG. 20, A shows grafts derived from unsorted precursor cells, CLSTN2-sorted precursor cells, and PTPRO-sorted precursor cells, respectively, and the typical maps of the grafts immunostained with human nuclei (HNA), TH and DAT. Here, the typical plots of immunostaining with TH of the 5-hydroxytryptaminergic neuronal marker gene 5-HT(B) or GABAergic neuronal marker gene GABA(D) and mDA marker gene are shown. Quantitative analysis was performed on the 5-HT+ neuron ratio (C) and GABA+ neuron ratio (E) in each graft group.

FIG. 21 shows the composition of the graft cell type. Here, FIG. 21A shows the VLMC isotype ratio per graft group as calculated using scRNA-seq data. FIG. 21B shows typical images of grafts derived from cell sorted by the combination of LMX1A and EN1, with immunostaining with 5-HT or GABA and TH (left). The ratios of 5-HT⁺ and GABA⁺ neurons in the grafts (right) were quantified. FIGS. 21C-21G show immunohistochemical staining of oligodendrocyte- or oligodendrocyte precursor cell-labeled Olig2 (FIG. 21C), astrocyte-labeled GFAP (FIG. 21D), VLMC-labeled COL1A1 (FIG. 21E), and hN in each group of grafts. The proportions of Olig2⁺ cells (FIG. 21F) and GFAP⁺ cells (FIG. 21G) were quantified.

FIG. 22 shows CLSTN2 or PTPRO-enriched neural precursor cells producing smaller grafts but denser dopaminergic innervation after transplantation. FIG. 22A shows that the immunostaining of transplanted neurons with hNCAM indicates the distributions of hNCAM⁺ fibers in the dorsal striatum (caudate putamen, CPu; illustration box i) and ventral striatum (lateral nucleus accumbens shell, LAcbSh; illustration box ii; olfactory tubercle, Tu; in illustration), and extension (box three). The white asterisk indicates a transplantation site. The scale bar corresponds to 500 μm. FIG. 22B shows the estimated graft volume by hN staining on Month 6. N=9 (unsorted), 7 (CLSTN2), and 8 (PTPRO). One-way analysis of variance was followed by Tukey's multiple comparison test. FIG. 22C shows a schematic diagram of TH-specific histological assessment and electrophysiological recording in surface marker-derived grafts. FIG. 22D shows immunostaining of tdT in TH+ neurons in CLSTN2- (left) or PTPRO-derived (right) grafts. The box region is enlarged on the right. The white arrow indicates the neuron that co-expresses tdT and TH. The scale bar corresponds to 20 μm. FIG. 22E shows serial coronal sections for graft immunostaining for tdT. The white asterisk indicates a transplantation site. The scale bar corresponds to 500 μm. FIG. 22F shows typical immunohistochemical images with tdT (representing TH) labeled in the graft. The scale bar corresponds to 500 μm. FIG. 22G shows the average gray values of tdT pixels quantifying the grafts in four random regions. One-way analysis of variance was followed by Tukey's multiple comparison test.

FIG. 23 shows graft fiber innervation across different brain regions and the synaptic integration of mDA neurons. FIG. 23A shows hNCAM⁺ fiber distribution and extension in the LMX1A⁺EN1⁺ group in Stage III or IV. The white asterisk indicates a transplantation site. The scale bar corresponds to 500 μm. FIG. 23B shows the examination of hNCAM fiber extension in different brain regions in each group. The scale bar corresponds to 500 μm. FIG. 23C shows the graft volumes in the LMX1A+EN1+ group in Stage III or IV on Month 6. N=5 (LMX1A⁺EN1⁺ in Stage III) and 8 (28 LMX1A⁺EN1⁺ in Stage IV). FIG. 23D shows grafts co-labeling human specific fibers STEM121 and TH. The scale bar corresponds to 100 μm. The illustration box represents a magnified view of the extended grafted fiber. The scale bar corresponds to 20 μm. FIG. 23E shows a typical immunohistochemical image of a CLSTN2-derived graft co-labeled with human-specific synaptophysin and TH. The box region is enlarged on the right. The white arrow indicates colocalization of the human-specific synaptophysin and TH along TH fibers. The scale bar corresponds to 20 μm.

FIG. 24 shows the electrophysiological recording of transplanted mDA neurons. FIGS. 24A and 24B show typical plots of current-induced APs (FIG. 24A) and single current-induced AP (FIG. 24B) from transplanted mDA neurons recorded by the whole-cell patch-clamp 5 months after transplantation. FIGS. 24C and 24D show resting membrane potentials (FIG. 24C), thresholds (FIG. 24D), and after hyperpolarization (AHP) (FIG. 24E) from transplanted mDA neurons. FIGS. 24F and 24G show statistics on the maximum frequency of ramp current-induced APs (100-300 pA for 2,000 ms) (FIG. 24F) and ramp current-induced APs of transplanted mDA neurons (FIG. 24G). Number of cells recorded, n=23 (unsorted), 11 (CLSTN2), and 18 (PTPRO). FIGS. 24H and 24I show the amplitudes of sIPSCs (FIG. 22H) and sEPSCs (FIG. 24I). FIG. 24J shows that an amphetamine-induced rotational behavior changes 6 months after transplantation. N=9 (LMX1A⁺EN1⁺ in Stage III) and 8 (LMX1A⁺EN1⁺ in Stage V).

FIG. 25 shows that CLSTN2 or PTPRO-rich neural precursor cells are integrated into the host circuit and exhibit higher therapeutic efficacy. FIGS. 25A-25D show typical trajectories recorded by the whole-cell patch-clamp for spontaneous action potential (SAP) (FIG. 25A) and SAP frequency (FIG. 25B), showing sags from transplanted mDA neurons (FIG. 25C) and statistics of sags (FIG. 25D). Number of recorded cells, n=24 (unsorted), 15 (CLSTN2), and 22 (PTPRO). One-way analysis of variance was followed by Tukey's multiple comparison test. FIG. 25E shows the typical trajectories of sIPSCs (top) and sEPSCs (bottom) in transplanted human mDA neurons 5 months after transplantation. FIGS. 25F-25G plot the frequencies of sIPSCs (FIG. 25F) and sEPSCs (FIG. 25G). Number of mice, n=4 (unsorted), 3 (CLSTN2), and 4 (PTPRO). Number of sEPSC cells recorded, n=16 (unsorted), 16 (CLSTN2), and 20 (PTPRO). Number of sIPSC cells recorded, n=16 (unsorted), 18 (CLSTN2), and 20 (PTPRO). FIGS. 25H and 25I show changes in the rotation behavior of amphetamine-induced PD mice 6 months after transplantation. The graft dose was 100,000 cells per mouse (FIG. 25H). N=5 (aCSF), 9 (unsorted), 11 (CLSTN2), and 9 (PTPRO). FIG. 25I shows a graft dose of 7,500 cells per mouse. The H9-CLSTN2-P2A-tdT cell line was used. N=4 (unsorted), and 3 (sorted). Dunnett's multiple comparison test and bidirectional ANOVA for comparison with the non-sorting group were used.

### DETAILED DESCRIPTION OF THE PREFERRED EMBODIMENTS

The embodiments of the invention of the present application will be illustrated by specific examples below. Those familiar with this technology may easily understand other advantages and effects of the invention of the present application from the content disclosed in the specification.

### Terms & Definitions

In the present application, the term “CD83” generally refers to the differentiation cluster 83, and may also be referred to as BLII or HB15. CD83 protein is a type I transmembrane protein belonging to the receptor immunoglobulin superfamily. The “CD83” may include full-length CD83, the truncations, functional fragments, different transcripts, splicing variants and subtypes of CD83, naturally occurring CD83, and artificially modified or mutated CD83 proteins. Genetic information on “CD83” can be found in Ensembl database under the Accession Number of ENSG00000112149, and protein information on “CD83” can be found in UniProt database under the Accession Number of Q01151. In the present application, the “CD83” may serve as a molecular marker for dopaminergic neural precursor cells.

In the present application, the term “CLSTN2” generally refers to the calmodulin 2, and may also be referred to as CDHR13, CS2, CSTN2 and FLJ39113. It is a membrane protein. The “CLSTN2” may include full-length CLSTN2, the truncations, functional fragments, different transcripts, splicing variants and subtypes of CLSTN2, naturally occurring CLSTN2, and artificially modified or mutated CLSTN2 proteins. Genetic information on “CLSTN2” can be found in Ensembl database under the Accession Number of ENSG00000158258, and protein information on “CLSTN2” can be found in UniProt database under the Accession Number of Q9H4D0. In the present application, the “CLSTN2” may serve as a molecular marker for dopaminergic neural precursor cells.

In the present application, the term “FLRT2” generally refers to the fibronectin leucine-rich repeating transmembrane protein 2, which is a type I transmembrane protein. The FLRT2 protein plays a role in cell-to-cell adhesion, cell migration, and axon guidance. The “FLRT2” may include full-length FLRT2, the truncations, functional fragments, different transcripts, splicing variants and subtypes of FLRT2, naturally occurring FLRT2, and artificially modified or mutated FLRT2 proteins. Genetic information on “FLRT2” can be found in Ensembl database under the Accession Number of ENSG00000185070, and protein information on “FLRT2” can be found in UniProt database under the Accession Number of 043155. In the present application, the “FLRT2” may serve as a molecular marker for dopaminergic neural precursor cells.

In the present application, the term “KITLG” generally refers to the ligand of the receptor-type protein tyrosine kinase KIT, and is also referred to as DFNA69, FPH2, Kit1, KL-1, MGF, SCF, SF and/or SLF. It plays a crucial role in regulating cell survival and proliferation, hematopoiesis, stem cell maintenance, gametogenesis, mast cell development, migration and function, as well as melanin formation. The “KITLG” may include full-length KITLG, the truncations, functional fragments, different transcripts, splicing variants and subtypes of KITLG, naturally occurring KITLG, and artificially modified or mutated KITLG proteins. Genetic information on “KITLG” can be found in Ensembl database under the Accession Number of ENSG00000049130, and protein information on “KITLG” can be found in UniProt database under the Accession Number of P21583. In the present application, the “KITLG” may serve as a molecular marker for dopaminergic neural precursor cells.

In the present application, the term “NTRK3” generally refers to the neurotrophic receptor tyrosine kinase 3, and may also be referred to as TRKC. NTRK3 is a membrane-binding receptor that can be involved in the development of the nervous system and heart. The “NTRK3” may include full-length NTRK3, the truncations, functional fragments, different transcripts, splicing variants and subtypes of NTRK3, naturally occurring NTRK3, and artificially modified or mutated NTRK3 proteins. Genetic information on “NTRK3” can be found in Ensembl database under the Accession Number of ENSG00000140538, and protein information on “NTRK3” can be found in UniProt database under the Accession Number of Q16288. In the present application, the “NTRK3” may serve as a molecular marker for dopaminergic neural precursor cells.

In the present application, the term “PTPRO” generally refers to a protein tyrosine phosphatase O-type receptor, and may also be referred to as GLEPP1, NPHS6, PTP-oc. PTP-U2 and PTPU2. It is a transmembrane protein. The “PTPRO” may include full-length PTPRO, the truncations, functional fragments, different transcripts, splicing variants and subtypes of PTPRO, naturally occurring PTPRO, and artificially modified or mutated PTPRO proteins. Genetic information on “PTPRO” can be found in Ensembl database under the Accession Number of ENSG00000151490, and protein information on “PTPRO” can be found in UniProt database under the Accession Number of Q16827. In the present application, the “PTPRO” may serve as a molecular marker for dopaminergic neural precursor cells.

In the present application, the term “LMX1A” generally refers to the LIM homeobox transcription factor 1α, and acts on the development of dopaminergic neurons during embryogenesis. The “LMX1A” may include full-length LMX1A, the truncations, functional fragments, different transcripts, splicing variants and subtypes of LMX1A, naturally occurring LMX1A, and artificially modified or mutated LMX1A proteins. Genetic information on “LMX1A” can be found in Ensembl database under the Accession Number of ENSG00000162761, and protein information on “LMX1A” can be found in UniProt database under the Accession Number of Q8TE12. In the present application, the “LMX1A” may serve as a molecular marker for dopaminergic neural precursor cells.

In the present application, the term “EN1” generally refers to the Engrailed homeobox, and mainly helps regulate the development of the dorsal midbrain and hindbrain. The “EN1” may include full-length EN1, the truncations, functional fragments, different transcripts, splicing variants and subtypes of EN1, naturally occurring EN1, and artificially modified or mutated EN1 proteins. Genetic information on “EN1” can be found in Ensembl database under the Accession Number of ENSG00000163064, and protein information on “EN1” can be found in UniProt database under the Accession Number of Q05925. In the present application, the “EN1” may serve as a molecular marker for dopaminergic neural precursor cells.

In the present application, in relation to a cell, the superscript “+” following a molecular marker (such as “CD83”, “CLSTN2”, “FLRT2”, “KITLG”, “NTRK3”, “PTPRO”, “LMX1A” and/or “EN1”) generally means that the cell is positive for this molecular marker. That is, the cell expresses this molecular marker. For example, CLSTN2⁺ in relation to a cell indicates that the cell is positive for CLSTN2. For example, the cell can express a CLSTN2 protein, or can transcribe CLSTN2 RNA. When a cell is positive for more than one marker, for example, when the expression of LMX1A⁺EN1⁺ is used, the cell is positive for both LMX1A and EN1. The term “positive” means that, in an experiment for determining the expression and/or activity of a molecular marker, the result is higher than the threshold or cut-off value of that assay test, for samples that are considered to reproducibly comprise a detectable level of the molecular marker.

Whether a cell is positive for a molecular marker can be determined by detecting the expression of the molecular marker (including a protein and/or nucleic acid) for the cell. In some cases, for a molecular marker (such as “CD83”, “CLSTN2”, “FLRT2”, “KITLG”, “NTRK3”, “PTPRO”, “LMX1A” and/or “EN1”), a molecule capable of specifically recognizing or binding to the molecular marker may be used, and the molecule may include a protein, a nucleic acid, a macromolecule and/or a micromolecule. For example, the molecule may be an antibody having a detectable marker and capable of specifically binding to a molecular marker protein. By mixing the antibody with a cell to be detected, if the antibody is detected with the capability of binding to the cell to be detected, it indicates that the cell is positive for the molecular marker. For another example, the molecule may be a probe with a detectable marker (for example, a fluorescent probe) and capable of specifically hybridizing a molecular marker nucleic acid, and if the expression of the detectable marker is detected, it indicates that the cell is positive for the molecular marker. Compared to a control cell or a negative cell, the expression and/or activity of the molecular marker can be qualified or quantified for the cell. In some cases, the expression of a target molecular marker may also be determined by means of the expression of a marker molecule by inserting a detectable marker molecule (for example, a fluorescent reporter gene) into the expression cassette of the target molecular marker.

In the present application, the term “marker molecule” generally refers to a substance capable of indicating the expression and/or activity of the molecular marker (such as “CD83”, “CLSTN2”, “FLRT2”, “KITLG”, “NTRK3”, “PTPRO”, “LMX1A” and/or “EN1”), and includes those capable of directly recognizing or binding to the molecular marker proteins or nucleic acids as defined, and those reflecting the expression and/or activity of the molecular marker protein or nucleic acid by means of their own expression and/or activity. For example, the marker molecule may be a radioisotope, a fluorophore, a chemiluminescent substance, a chromophore, an antibody, an enzyme, a substrate of an enzyme, a cofactor of an enzyme, an inhibitor of an enzyme, a chromophore, a dye, a metal ion, a metsol, a ligand (such as a biotin, avidin, streptavidin or hapten) or the like.

In the present application, the term “dopaminergic neural precursor cells” generally refers to cells capable of proliferating and/or differentiating into dopaminergic neurons in vitro or in vivo. The dopaminergic neural precursor cells may be derived from midbrain ventral neural cells and can be differentiated from pluripotent stem cells. The dopaminergic neural precursor cells may also be differentiated or reprogrammed from other cell types. Common dopaminergic neural precursor cell markers may include LMX1A, EN1, OTX2, and/or FOXA2.

In the present application, the term “dopaminergic neurons” generally refers to cells containing and releasing dopamine as a neurotransmitter. Common dopaminergic neural cell markers may include a tyrosine hydroxylase (TH), a dopamine transporter (DAT), an activating transcription factor FOXA2, a G-protein-regulated potassium channel GIRK2, a transcription factor Nurr1, a transcription factor EN1, and/or a transcription factor LMX1B. In the present application, the term “dopaminergic neural cells” may be used interchangeably with the “dopaminergic nerve precursor cells” or the “dopaminergic neurons”. The dopaminergic neural cells may be, but not limited to, dopaminergic neural progenitor cells, or dopaminergic neural precursor cells, or mature dopaminergic neurons. The dopaminergic neural cells described in the present application may be midbrain dopaminergic neural cells. As used herein, the term “midbrain dopaminergic (mDA) neural cells” generally refers to, but is not limited to, dopaminergic neural cells observed in a midbrain region, for example, in the midbrain ventral region. In addition, the mDA neural cells may be specific to an A9 region. The “A9 region” is a midbrain ventral region, which corresponds to the dense part of the substantia nigra. The A9 region is a region with a large amount of dopaminergic neural cells found, and is correlated with the control of motor function. Especially in patients with PD, the dopaminergic neural cells specifically degenerate in this region.

In the present application, the term “cell population” may include, but is not limited to, human stem cells: the progenitor cells or precursors of the human stem cells: dopaminergic neural progenitor cells derived from human stem cells or precursors and/or dopaminergic neural precursor cells or mature dopaminergic neurons, and neural derivatives derived therefrom. Specifically, examples of human stem cells or precursor cells may include, but are not limited to, embryonic stem cells, embryonic germ cells, embryonic cancer cells, induced pluripotent stem cells (iPSCs), adult stem cells, and fetal cells.

In the present application, the term “pluripotent stem cells” generally refers to a class of cells that have the potential to differentiate into any cell in a human body. The pluripotent stem cells may be derived from fertilized eggs or somatic cells, and the somatic cells may include blood cells, urine cells, skin cells, and/or umbilical cord blood cells. Depending on their origin, the pluripotent stem cells may include human embryonic stem cells (derived from fertilized eggs) and human induced pluripotent stem cells (somatic cells). The pluripotent stem cells have the ability to proliferate sustainably and differentiate into a variety of cells.

In the present application, the term “candidate cells” generally refers to cells to be identified whether they are dopaminergic nerve precursor cells. The “candidate cells” may be neural precursor cells.

In the present application, the term “neural precursor cells” generally refers to undifferentiated precursor cells that has not expressed terminal differentiation features, and can be proliferated and/or differentiated into mature neuron cells. “Progenitor cells”, “precursors”, and “precursor cells” are used interchangeably herein. Depending on the neurotransmitters of the neurons produced by differentiation, the neural precursor cells may include cholinergic precursor cells, adrenergic precursor cells, GABAergic precursor cells, glutamatergic precursor cells, dopaminergic precursor cells, serotonergic precursor cells, and/or purinergic precursor cells. Depending on the synchronization of differentiation regions, the neural precursor cells may comprise midbrain ventral floor plate neural precursor cells, hindbrain floor plate neural precursor cells, and/or midbrain floor plate neural precursor cells.

In the present application, the term “neural cells” refers to the cells constituting the nervous system, and may comprise neural progenitor cells, precursor cells, stem cells, immature neurons and/or mature neurons. In some embodiments, the neural cells may be used in the same sense as neurons.

In the present application, the term “modification” generally refers to marking the candidate cells, for example, using marker molecules. The modification may include the modification of the candidate cells at a gene level, at an RNA level, or at a protein level. For example, the modification may refer to the insertion of a reporter gene in a molecular marker gene expression box.

In the present application, the term “Parkinson's disease (PD)” generally refers to a group of diseases related to dopamine deficiency in the basal ganglia (which is the part of the brain that controls movement). Symptoms include tremor, bradykinesia (very slow movement), flexed position, postural instability, and stiffness. The diagnosis of Parkinson's disease requires the presence of at least two of these symptoms, one of which must be tremor or bradykinesia. The Parkinson's disease includes idiopathic or typical Parkinson's disease and Parkinson's superposition syndrome (atypical Parkinson's disease). Typically, Parkinson's disease involves dysfunction and death of important neural cells primarily in a brain region called substantia nigra. Many of these important neural cells produce dopamine, and when these neurons die one after another, the amount of dopamine in the brain decreases, making it impossible for a person to control movement normally. The cluster of symptoms experienced by individuals varies from person to person. The main motor symptoms of Parkinson's disease include: tremors of the hands, arms, legs, jaw, and face, bradykinesia or slow movement, rigidity or stiffness of the limbs and trunk, and postural instability or impaired balance and coordination. The rate of progression of the Parkinson's disease can be scored and quantified by means of the total Unified Parkinson's Disease Rating Scale (total UPDRS).

In the present application, the term “midbrain” refers to the region between the forebrain (anterior) and hindbrain (posterior) of the vertebrate brain under development. The midbrain region gives rise to many regions of the brain, including but not limited to reticular formation, which is part of the tegmentum, a region of the brainstem that influences motor functions, the crus cerebri, which is made up of nerve fibers connecting the cerebral hemispheres to the cerebellum, and a large pigmented nucleus called the substantia nigra. A unique feature of the developing midbrain is the co-expression of the floor plate marker FOXA2 and the roof plate marker LMX1A.

In the present application, the term “dopamine neurons” or “dopaminergic neurons” generally refers to cells containing and able to release dopamine. “Midbrain dopamine neurons” or “mDA” refers to neuronal cells in the midbrain structure that are presumed to contain and release dopamine and neuronal cells in the midbrain structure that contain and release dopamine.

In the present application, the term “dopamine” generally refers to a catecholamine neurotransmitter produced and released by dopaminergic neurons.

In the present application, the term “sonic hedgehog (SHH or Shh)” refers to a protein that is one of at least three proteins in the mammalian signaling pathway family, including hedgehog, desert hedgehog (DHH) and Indian hedgehog (IHH). Shh interacts with at least two transmembrane proteins by interacting with transmembrane molecules Patched (PTC) and Smoothened (SMO). Shh typically binds to PCT, which then allows the activation of SMO as a signal transducer. In the absence of SHH, PTC typically inhibits SMO, which in turn activates a transcriptional repressor, such that the transcription of certain genes does not occur. When Shh is present and binds to PTC, PTC cannot interfere with the functioning of SMO. With SMO uninhibited, certain proteins are able to enter the nucleus and act as transcription factors allowing certain genes to be activated.

In the present application, the term “activator of Sonic hedgehog (SHH) signaling” refers to any molecule or compound that can activate a SHH signaling pathway, including a molecule or compound that binds to PCT or a Smoothened agonist and the like. For example, the protein Sonic hedgehog (SHH) C25II, SAG and the small molecule Smoothened agonist purmorphamine may be included.

In the present application, the term “differentiation” generally refers to a process whereby an unspecialized stem cell acquires the features of a specialized cell (for example, a specific type of neuron, blood cell, and heart, liver, or muscle cell). Differentiation is controlled by the interaction of a cell's gene with the physical and chemical conditions outside the cell, usually through signaling pathways involving proteins embedded in the cell surface. In the present application, the term “differentiation” as used with respect to cells in a differentiating cell system refers to the process by which cells differentiate from one cell type to another cell type.

In the present application, the terms “isolation”, “sorting” and “screening” are used interchangeably, and when used for cells, generally refer to the isolation of a particular cell subpopulation from a mixed cell population according to the characteristics of the cell. For example, in the present application, the isolation refers to the isolation of cells having one or more of the following features from the cell population: CLSTN2⁺, PTPRO⁺, NTRK3⁺, FLRT2⁺, KITLG⁺, CD83⁺, and/or LMX1A⁺EN1⁺ In addition to cells having the above features, the cell population to be isolated typically comprises other cells that do not have the above features. For example, the cell population may include heterogeneous neural precursor cells, or undifferentiated neural precursor cells (for example, pluripotent stem cells), or completely or partially differentiated neural precursor cells. Common methods for cell isolation may include methods based on immunorecognition properties and/or methods based on the physical properties of cells. For example, the methods for isolation may include flow cytometry sorting, immunomagnetic cell sorting, and/or density gradient centrifugation.

In the present application, the term “enrichment” generally refers to increasing the proportion of cells in a cell population with certain common features. The enrichment may be achieved by isolating the cells having the common features as defined from cells not having the common features.

### DETAILED DESCRIPTION OF THE INVENTION

### Methods for Identification, Isolation and Enrichment

In an aspect, the present application provides a method for identifying dopaminergic neural precursor cells, comprising: determining whether candidate cells have one or more of the following features: CLSTN2⁺, PTPRO⁺, NTRK3⁺, FLRT2⁺, KITLG⁺, CD83⁺, and/or LMX1A⁺EN1⁺; and identifying cells having the features as the dopaminergic neural precursor cells. In the present application, if the candidate cells have one or more of the following features: CLSTN2⁺, PTPRO⁺, NTRK3⁺, FLRT2⁺, KITLG⁺, CD83⁺, and/or LMX1A⁺EN1⁺, the candidate cells can be identified as the dopaminergic neural precursor cells. In the present application, the candidate cells may be derived from pluripotent stem cells, for example, human pluripotent stem cells, and for another example, embryonic stem cells and induced pluripotent stem cells. For example, the induced pluripotent stem cells may be derived from autologous cells or allogeneic cells. In the present application, the candidate cells may be derived from midbrain ventral nerve tissues.

In the present application, the candidate cells may be an appropriately differentiated population of neural precursor cell population, which is generally heterogeneous. The heterogeneous neural precursor cell population is a cell population containing two or more types of neural precursor cells, or even neuroblasts and/or neurons. In the present application, the candidate cells may be pluripotent stem cells (for example, human pluripotent stem cells), which may be differentiated into neural precursor cells in vivo (for example, transplanted into the subject's brain) or in vitro, under certain conditions. In the present application, the pluripotent stem cells may be human embryonic stem cells and/or human induced pluripotent stem cells. In the present application, the cells (for example, pluripotent stem cells) to be differentiated may be derived from rodent cells, primate cells, or human cells. For example, the cells (for example, pluripotent stem cells) to be differentiated may be derived from a normal human or a patient with the symptoms of Parkinson's disease.

Methods for differentiation from stem cells or other types of cells into neural precursor cells are known, in which, for example, small molecules, growth factor proteins, and other growth conditions are utilized to promote the transformation of cells from a pluripotent state to a more mature or specialized cellular outcome (for example, central nervous system cells, neural cells, brain cells in the floor plate, or dopaminergic neural cells). In some cases, the differentiation may comprise contacting the cells or cell population to be differentiated, with an ALK inhibitor, a sonic hedgehog SHH signaling activator and/or a GSK-3 inhibitor. Contacting may be performed under a condition enabling differentiation of the cell population into midbrain floor plate precursor cells.

Depending on the differentiation conditions and differentiation methods, the differentiation time for the cells in the pluripotent state may be at least about 10 days, about 12 days, about 13 days, about 14 days, 15 days, 18 days, 21 days, 25 days, 28 days, 30 days, 35 days or more, for example, 14 days or 21 days. Those skilled in the art are able to determine, under different differentiation conditions and methods, whether neural precursor cells derived from the pluripotent stem cells can be used to determine the presence of one or more of the following features: CLSTN2⁺, PTPRO⁺, NTRK3⁺, FLRT2⁺, KITLG⁺, CD83⁺ and/or LMX1A⁺EN1⁺.

In another aspect, the present application provides a method for isolating dopaminergic neural precursor cells, comprising: (a) providing a neural precursor cell population, and (b) isolating, from the neural precursor cell population, cells having one or more of the following features: CLSTN2⁺, PTPRO⁺, NTRK3⁺, FLRT2⁺, KITLG⁺, CD83⁺, and/or LMX1A⁺EN1⁺.

In another aspect, the present application provides a method for enriching dopaminergic neural precursor cells, comprising: (a) providing a neural precursor cell population, (b) determining whether cells in the neural precursor cell population have one or more of the following features: CLSTN2⁺, PTPRO⁺, NTRK3⁺, FLRT2⁺, KITLG⁺, CD83⁺, and/or LMX1A⁺EN1⁺, and (c), enriching the cells having any one or more of the above features.

In the present application, the isolation and/or enrichment may include the use of reagents capable of specifically recognizing and/or binding to molecular markers (for example, CLSTN2, PTPRO, NTRK3, FLRT2, KITLG, CD83, and/or a combination of LMX1A and EN1). The reagents may be proteins, for example, antibodies or antigen-binding fragments thereof, and affinity ligands, which may be labeled with fluorescein (for flow sorting) or used with magnetic beads (for magnetic bead sorting). When a cell population to be isolated is mixed with the proteins, the cells having one or more of the above features specifically bind to the reagent, and then are distinguished from cells that do not have the above properties, by means of differences in properties (for example, molecular weight, polarity, charge, fluorescence wavelength or the like). The reagents may be nucleic acid molecules capable of specifically recognizing and/or amplifying molecular markers (for example, CLSTN2, PTPRO, NTRK3, FLRT2, KITLG, CD83, and/or a combination of LMX1 A and EN1), and these nucleic acid molecules may carry marker genes. When the cell population to be isolated is mixed with the nucleic acid molecules, the nucleic acid molecules recognize and/or bind to cells having one or more of the above features, and during the gene expression of the molecular marker, the marker genes exist to distinguish these cells from cells that do not have the above features.

In the present application, the cell population of the present application may also be isolated and/or enriched using a non-antibody-based purification method, which includes, but is not limited to, size selection (for example, by density gradient, FACS, or MACS), application of a labeled ligand to a cell surface receptor, or the use of enhancer-promoter reporter gene expression or of labeled surface markers.

The neural precursor cells identified, isolated and/or enriched in the present application may be further differentiated into neurons, and the neural cells may comprise at least 10% or more (for example, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more or above) of midbrain dopaminergic neurons.

In another aspect, the present application provides a method for preparing a cell product, comprising isolating and/or enriching neural precursor cells having one or more of the following features: CLSTN2⁺, PTPRO⁺, NTRK3⁺, FLRT2⁺, KITLG⁺, CD83⁺, and/or LMX1A⁺EN1⁺.

In the present application, the cell product prepared by the method of the present application comprises neural precursor cells having one or more of the following features: CLSTN2⁺, PTPRO⁺, NTRK3⁺, FLRT2⁺, KITLG⁺, CD83⁺, and/or LMX1A⁺EN1⁺. When the cell product is further differentiated into neurons in vivo or in vitro, the neurons may comprise at least 10% or more (for example, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more or above) midbrain dopaminergic neurons. The cell products of the present application have a clear and stable cell composition after in vivo (transplantation) or in vitro differentiation. For example, in a specific embodiment, grafts sorted by different molecular markers have a similar neuronal composition, and are mainly composed of three different subtypes of dopaminergic neurons and one subtype of glutamatergic neurons.

In another aspect, the cell product may be further expanded and proliferated in vivo or in vitro to obtain a cell preparation, and the neural precursor cells have one or more of the following features: CLSTN2⁺, PTPRO⁺, NTRK3⁺, FLRT2⁺, KITLG⁺, CD83⁺, and/or LMX1A⁺EN1⁺. The cell preparation may also comprise dopaminergic neural cells.

For cell products prepared by means of any method (including the method of the present application and/or methods known in the prior art), the present application further provides a method for evaluating these cell products. The method comprises: detecting the cell product for a proportion of cells having one or more of the following features: CLSTN2⁺, PTPRO⁺, NTRK3⁺, FLRT2⁺, KITLG⁺, CD83⁺, and/or LMX1A⁺EN1⁺. Based on the proportion (for example, 1%-10%, 10%-20% or more than 20%) of cells having one or more of the above features, the quality of the cell product may be evaluated, the post-transplantation therapeutic effect of the cell product may be predicted, or guidance may be provided for the administration dose and regimen of the cell product. In general, when the proportion of cells in the cell product having one or more of the above features is high (for example, greater than 10%), the administration dose or frequency of the cell product may be reduced. In general, when the proportion of cells in the cell product having one or more of the above features is low (for example, less than 10%), the administration dose or frequency of the cell product may be increased.

The present application further provides a method for optimizing a preparation process for a cell product. The method comprises: detecting the cell product for a proportion of cells having one or more of the following features: CLSTN2⁺, PTPRO⁺, NTRK3⁺, FLRT2⁺, KITLG⁺, CD83⁺, and/or LMX1A⁺EN1⁺, and guiding the preparation process of the cell product according to the proportion. For example, when the proportion of cells in the cell product having one or more of the above features is low (for example, less than 10%), it can be considered that the preparation process of the cell product needs to be optimized. The preparation process may comprise generation, differentiation, isolation, and/or purification of the cell product.

The present application further provides a method for controlling quality of prepared dopaminergic neural precursor cells, comprising the following steps: a) detecting the prepared cells for a proportion of cells having one or more of the following features: CLSTN2⁺, PTPRO⁺, NTRK3⁺, FLRT2⁺, KITLG⁺, CD83⁺, and/or LMX1A⁺EN1⁺; and b) if the proportion detected in step a) is at least about 10% (for example, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more or above), determining that the quality of the prepared dopaminergic neural precursor cells is acceptable. For example, if the proportion detected in step a) is at least 10%, the prepared dopaminergic neural precursor cells meet the requirements for subsequent further differentiation in vivo (transplantation) or in vitro to obtain grafts. Here, for example, the prepared dopaminergic neural precursor cells can achieve a therapeutic effect.

On the contrary, if the proportion detected in step a) is less than 10%, it may be necessary to improve the quality of the prepared dopaminergic neural precursor cells by using the method for optimizing the preparation process of the cell product.

### Molecular Marker

For each type of molecular marker, different detection methods may be used to determine whether the molecular marker is positive according to the expression and activity characteristics of the molecular marker, for example, according to whether the molecular marker is expressed on the cell surface or in the cell, and whether a expression product is a membrane-bound protein or a free protein. The detection methods include, but are not limited to, immunohistochemical analysis, PCR, RT-PCR, in situ hybridization, southern blotting, western blotting, northern blotting, spectrophotometry, gene chip, flow cytometry (FACS), protein chip, DNA sequencing, and ELISA.

For the provided cells or cell population, the method comprises determining whether the cells (for example, candidate cells) have the following feature: CLSTN2⁺. For example, the method comprises detecting the candidate cells for the expression level of a CLSTN2 protein, the activity level of the CLSTN2 protein, the expression level of a CLSTN2 nucleic acid, and/or the activity level of the CLSTN2 nucleic acid. In some cases, the method may comprise the use of primers capable of specifically amplifying nucleic acid molecules encoding CLSTN2. The primers may be a pair of primers. In addition, the method may comprise the use of probes capable of specifically recognizing nucleic acid molecules encoding CLSTN2. The probes may be able to bind to a CLSTN2 nucleotide sequence or a fragment thereof, instead of another nucleotide sequence. The probes may have detectable signals. In other cases, the method may include the use of reagents capable of specifically recognizing the CLSTN2 protein and/or reagents capable of measuring the activity of the CLSTN2 protein, such as the antibody and/or ligand of the CLSTN2 protein and/or fragments thereof.

For the provided cells or cell population, the method comprises determining whether the cells (for example, candidate cells) have the following feature: PTPRO⁺. For example, the method comprises detecting the candidate cells for the expression level of a PTPRO protein, the activity level of the PTPRO protein, the expression level of a PTPRO nucleic acid, and/or the activity level of the PTPRO nucleic acid. In some cases, the method may comprise the use of primers capable of specifically amplifying nucleic acid molecules encoding PTPRO. The primers may be a pair of primers. In addition, the method may comprise the use of probes capable of specifically recognizing nucleic acid molecules encoding PTPRO. The probes may be able to bind to a PTPRO nucleotide sequence or a fragment thereof, instead of another nucleotide sequence. The probes may have detectable signals. In other cases, the method may include the use of reagents capable of specifically recognizing the PTPRO protein and/or reagents capable of measuring the activity of the PTPRO protein, such as the antibody and/or ligand of the PTPRO protein and/or fragments thereof.

For the provided cells or cell population, the method comprises determining whether the cells (for example, candidate cells) have the following feature: NTRK3⁺. For example, the method comprises detecting the candidate cells for the expression level of an NTRK3 protein, the activity level of the NTRK3 protein, the expression level of a NTRK3 nucleic acid, and/or the activity level of then NTRK3 nucleic acid. In some cases, the method may comprise the use of primers capable of specifically amplifying nucleic acid molecules encoding NTRK3. The primers may be a pair of primers. In addition, the method may comprise the use of probes capable of specifically recognizing nucleic acid molecules encoding NTRK3. The probes may be able to bind to an NTRK3 nucleotide sequence or a fragment thereof, instead of another nucleotide sequence. The probes may have detectable signals. In other cases, the method may include the use of reagents capable of specifically recognizing the NTRK3 protein and/or reagents capable of measuring the activity of the NTRK3 protein, such as the antibody and/or ligand of the NTRK3 protein and/or fragments thereof.

For the provided cells or cell population, the method comprises determining whether the cells (for example, candidate cells) have the following feature: FLRT2. For example, the method comprises detecting the candidate cells for the expression level of an FLRT2 protein, the activity level of the FLRT2 protein, the expression level of an FLRT2 nucleic acid, and/or the activity level of the FLRT2 nucleic acid. In some cases, the method may comprise the use of primers capable of specifically amplifying nucleic acid molecules encoding FLRT2. The primers may be a pair of primers. In addition, the method may comprise the use of probes capable of specifically recognizing nucleic acid molecules encoding FLRT2. The probes may be able to bind to an FLRT2 nucleotide sequence or a fragment thereof, instead of another nucleotide sequence. The probes may have detectable signals. In other cases, the method may include the use of reagents capable of specifically recognizing the FLRT2 protein and/or reagents capable of measuring the activity of the FLRT2 protein, such as the antibody and/or ligand of the FLRT2 protein and/or fragments thereof.

For the provided cells or cell population, the method comprises determining whether the cells (for example, candidate cells) have the following feature: KITLG⁺. For example, the method comprises detecting the candidate cells for the expression level of a KITLG protein, the activity level of the KITLG protein, the expression level of a KITLG nucleic acid, and/or the activity level of the KITLG nucleic acid. In some cases, the method may comprise the use of primers capable of specifically amplifying nucleic acid molecules encoding KITLG. The primers may be a pair of primers. In addition, the method may comprise the use of probes capable of specifically recognizing nucleic acid molecules encoding KITLG. The probes may be able to bind to a KITLG nucleotide sequence or a fragment thereof, instead of another nucleotide sequence. The probes may have detectable signals. In other cases, the method may include the use of reagents capable of specifically recognizing the KITLG protein and/or reagents capable of measuring the activity of the KITLG protein, such as the antibody and/or ligand of the KITLG protein and/or fragments thereof.

For the provided cells or cell population, the method comprises determining whether the cells (for example, candidate cells) have the following feature: CD83⁺. For example, the method comprises detecting the candidate cells for the expression level of a CD83 protein, the activity level of the CD83 protein, the expression level of a CD83 nucleic acid, and/or the activity level of the CD83 nucleic acid. In some cases, the method may comprise the use of primers capable of specifically amplifying nucleic acid molecules encoding CD83. The primers may be a pair of primers. In addition, the method may comprise the use of probes capable of specifically recognizing nucleic acid molecules encoding CD83. The probes may be able to bind to a CD83 nucleotide sequence or a fragment thereof, instead of another nucleotide sequence. The probes may have detectable signals. In other cases, the method may include the use of reagents capable of specifically recognizing the CD83 protein and/or reagents capable of measuring the activity of the CD83 protein, such as the antibody and/or ligand of the CD83 protein and/or fragments thereof.

For the provided cells or cell population, the method comprises determining whether the cells (for example, candidate cells) have the following feature: LMX1A⁺EN1⁺, that is, whether the cells are positive for both LMX1A and EN1. For example, the method comprises detecting the candidate cells for the expression level of an LMX1A protein, the activity level of the LMX1A protein, the expression level of an LMX1A nucleic acid, and/or the activity level of the LMX1A nucleic acid, and detecting the candidate cells for the expression level of an EN1 protein, the activity level of the EN1 protein, the expression level of an EN1 nucleic acid, and/or the activity level of the EN1 nucleic acid. In some cases, the method may comprise the use of primers capable of specifically amplifying nucleic acid molecules encoding LMX1A or EN1. The primers may be a pair of primers. In addition, the method may comprise the use of probes capable of specifically recognizing nucleic acid molecules encoding LMX1A or EN1. The probes may be able to bind to an LMX1A or EN1 nucleotide sequence or a fragment thereof, instead of another nucleotide sequence. The probes may have detectable signals. In other cases, the method may include the use of reagents capable of specifically recognizing the LMX1A or EN1 protein and/or reagents capable of measuring the activity of the LMX1A or EN1 protein, such as the antibody and/or ligand of the LMX1A or EN1 protein and/or fragments thereof.

In the present application, the expression of molecular marker (CLSTN2, PTPRO, NTRK3, FLRT2, KITLG, CD83, and/or the combination of LMX1A and EN1) may include the expression level of the molecular marker in the cells (for example, candidate cells) and/or the proportion of the number of cells positive for the molecular marker with respect to the number of cells in the overall cell population. In some embodiments, with different detection methods, when the content of a molecular marker in a cell is detected higher than the detection limit or threshold, the cell may be considered positive for the molecular marker. Or, in a cell population, when the proportion of the number of cells positive for the molecular marker with respect to the number of cells in the overall cell population is detected, it can be considered as the proportion of dopaminergic neural precursor cells in the cell population, and can also be used to speculate on the proportion of terminal dopaminergic neurons obtained by differentiation (including in vivo differentiation and in vitro differentiation).

In another aspect, the present application provides use of a CLSTN2⁺ indicator, a PTPRO⁺ indicator, an NTRK3⁺ indicator, an FLRT2⁺ indicator, KITLG⁺ indicator, CD83⁺ indicator and/or an LMX1A⁺EN1⁺ indicator CLSTN2⁺ indicator in preparation of a cell product, wherein the cell product comprises dopaminergic neural precursor cells. The indicator may be used to indicate or detect the activity and/or level of the molecular marker. In the present application, the indicator may include proteins, nucleic acids and/or small molecules. For example, the indicator may comprise a reagent capable of specifically binding to a molecular marker protein and/or a reagent capable of measuring the activity of the molecular marker protein. For another example, the indicator may comprise primers capable of specifically amplifying a nucleic acid molecule encoding a molecular marker and/or probes capable of specifically recognizing the nucleic acid molecule encoding the molecular marker. For example, the indicator may be an antibody capable of specifically binding to CLSTN2, PTPRO, NTRK3, FLRT2, KITLG, CD83, or a combination of LMX1 A and EN1, or an antigen-binding protein thereof.

In another aspect, the present application provides a quality control kit for preparing dopaminergic neural precursor cells, comprising a quality control reagent capable of determining whether candidate cells have one or more of the following features: CLSTN2⁺, PTPRO⁺, NTRK3⁺, FLRT2⁺, KITLG⁺, CD83⁺, and/or LMX1A⁺EN1⁺. In some cases, the quality control reagent may include the CLSTN2+ indicator, PTPRO+ indicator, NTRK3+ indicator, FLRT2+ indicator, KITLG+ indicator, CD83+ indicator and/or LMX1A+EN1+ indicator of the present application.

In some cases, the kit may further comprise a reagent capable of culturing and/or preserving the candidate cells. The reagent may be a cell culture medium, for example, a neural precursor cell culture medium and/or a neural precursor cell differentiation culture medium. In the kit, the reagent capable of culturing and/or preserving the candidate cells may be packaged independently with the quality control reagent.

In some cases, the kit may further comprise the candidate cells of the present application. The candidate cells may be the subsequent cells of the present application, for example, the neural precursor cells (for example, the neural precursor cells derived from human pluripotent stem cells).

### Cell Product

The present application provides an isolated or enriched dopaminergic neural precursor cell population, which expresses one or more of the following dopaminergic neural precursor cell markers: CLSTN2⁺, PTPRO⁺, NTRK3⁺, FLRT2⁺, KITLG⁺, CD83⁺, and/or LMX1A⁺EN1⁺.

In some cases, the isolated or enriched dopaminergic neural precursor cell population has a feature of CLSTN2⁺.

In some cases, the isolated or enriched dopaminergic neural precursor cell population has a feature of PTPRO⁺.

In some cases, the isolated or enriched dopaminergic neural precursor cell population has a feature of NTRK3⁺.

In some cases, the isolated or enriched dopaminergic neural precursor cell population has a feature of FLRT2⁺.

In some cases, the isolated or enriched dopaminergic neural precursor cell population has a feature of KITLG⁺.

In some cases, the isolated or enriched dopaminergic neural precursor cell population has a feature of CD83⁺.

In some cases, the isolated or enriched dopaminergic neural precursor cell population has a feature of LMX1A⁺EN1⁺.

In another aspect, the present application provides a dopaminergic neural precursor cell population, wherein at least about 10%, 20%, 30%, 40%, 50%, 60%, 70%, 80%, or 90% of cells express one or more of the following dopaminergic neural precursor cell markers: CLSTN2⁺, PTPRO⁺, NTRK3⁺, FLRT2⁺, KITLG⁺, CD83⁺, and/or LMX1A⁺EN1⁺.

In some cases, in the dopaminergic neural precursor cell population, at least about 10%, 20%, 30%, 40%, 50%, 60%, 70%, 80%, or 90% of cells express CLSTN2.

In some cases, in the dopaminergic neural precursor cell population, at least about 10%, 20%, 30%, 40%, 50%, 60%, 70%, 80%, or 90% of cells express PTPRO.

In some cases, in the dopaminergic neural precursor cell population, at least about 10%, 20%, 30%, 40%, 50%, 60%, 70%, 80%, or 90% of cells express NTRK3.

In some cases, in the dopaminergic neural precursor cell population, at least about 10%, 20%, 30%, 40%, 50%, 60%, 70%, 80%, or 90% of cells express FLRT2.

In some cases, in the dopaminergic neural precursor cell population, at least about 10%, 20%, 30%, 40%, 50%, 60%, 70%, 80%, or 90% of cells express KITLG.

In some cases, in the dopaminergic neural precursor cell population, at least about 10%, 20%, 30%, 40%, 50%, 60%, 70%, 80%, or 90% of cells express CD83.

In some cases, in the dopaminergic neural precursor cell population, at least about 10%, 20%, 30%, 40%, 50%, 60%, 70%, 80%, or 90% of cells express LMX1A and EN1.

In some cases, in the dopaminergic neural precursor cell population, at least about 40%, 50%, 60%, 70%, 80%, or 90% of cells express one or more of the following dopaminergic neural precursor cell markers: CLSTN2⁺, PTPRO⁺, NTRK3⁺, FLRT2⁺, KITLG⁺, CD83⁺, and/or LMX1A⁺EN1⁺.

In some cases, in the dopaminergic neural precursor cell population, at least about 40%, 50%, 60%, 70%, 80%, or 90% of cells express CLSTN2.

In some cases, in the dopaminergic neural precursor cell population, at least about 40%, 50%, 60%, 70%, 80%, or 90% of cells express PTPRO.

In some cases, in the dopaminergic neural precursor cell population, at least about 40%, 50%, 60%, 70%, 80%, or 90% of cells express NTRK3.

In some cases, in the dopaminergic neural precursor cell population, at least about 40%, 50%, 60%, 70%, 80%, or 90% of cells express PTPRO⁺.

In some cases, in the dopaminergic neural precursor cell population, at least about 40%, 50%, 60%, 70%, 80%, or 90% of cells express FLRT2.

In some cases, in the dopaminergic neural precursor cell population, at least about 40%, 50%, 60%, 70%, 80%, or 90% of cells express CD83.

In some cases, in the dopaminergic neural precursor cell population, at least about 40%, 50%, 60%, 70%, 80%, or 90% of cells express LMX1A and EN1.

In another aspect, the present application provides a cell product, comprising dopaminergic neural precursor cells obtained according to the method of the present application.

In another aspect, the present application provides a cell product, comprising the dopaminergic neural precursor cell population of the present application.

In another aspect, the present application provides a graft composition prepared from in vivo or in vitro differentiation of the dopaminergic neural precursor cell population of the present application.

In another aspect, the present application provides a pharmaceutical composition, comprising the dopaminergic neural precursor cell population of the present application or the cell product of the present application.

In some cases, the pharmaceutical composition may further comprise a pharmaceutically acceptable adjuvant.

### Therapeutic Method or Use of Prepared Medicament

In another aspect, the present application provides use of the cell product of the present application in screening of medicaments for prevention, treatment or alleviation of nervous system diseases or disorders. For example, the nervous diseases or disorders may comprise neuronal degeneration-related diseases or disorders (for example, Parkinson's disease). In the present application, the cell product may be differentiated into dopaminergic neurons, which are then brought into contact with a medicament to be screened. If the medicament to be screened has one or more of the following properties: (1) capability of preventing the death of the dopaminergic neurons, (2) capability of promoting the survival of the dopaminergic neurons, and (3) capability of improving the metabolism of the dopaminergic neurons, the medicament to be screened is selected as a medicament capable of preventing, treating, or alleviating the nervous diseases or disorders.

In another aspect, the present application provides a method for preventing, treating, or alleviating neurological diseases or disorders. The method comprises the following steps: identifying whether candidate cells have one or more of the following features: CLSTN2⁺, PTPRO⁺, NTRK3⁺, FLRT2⁺, KITLG⁺, CD83⁺, and/or LMX1A⁺EN1⁺; selecting cells having the features: and administering an effective dose of the cells having the features to a subject in need thereof.

In another aspect, the present application provides a pharmaceutical composition, comprising neural precursor cells having one or more of the following features: CLSTN2⁺, PTPRO⁺, NTRK3⁺, FLRT2⁺, KITLG⁺, CD83⁺, and/or LMX1A⁺EN1⁺. In some embodiments, the pharmaceutical composition further comprises a pharmaceutically acceptable adjuvant. In some embodiments, the cells can be differentiated into neural cells, which comprise at least 10% (for example, at least 15%, 20%, 25%, 30%, 35%, 40%, 45%, 50%, 55%, 60%, 65%, 70%, 75%, 80%, 85%, 90%, 95% or higher) of dopaminergic neural cells. In some embodiments, the differentiation comprises in vitro differentiation and in vivo differentiation.

According to an expected method, the dose of the pharmaceutical composition, the type of pharmaceutical preparation, and the route and time of administration may vary according to the condition, body weight, and degree of disease of a subject, and may be appropriately selected by those skilled in the art.

The pharmaceutical composition or cell product of the present application is administered at a pharmaceutically effective dose. The “pharmaceutically effective dose” refers to an amount sufficient to treat a disease at a reasonable benefit/risk ratio applicable to medical treatment or improvement, and the effective dose level may be determined according to elements including the following kinds: the disease, disease severity, age and gender of a subject, medicament activity, the sensitivity of the subject to the medicament, administration time, administration route, discharge rate, duration of treatment, and a medicament used concomitantly and other elements known in the medical field. The term “subject” generally refers to a subject in need of treatment and, more specifically, to a mammal, for example, a human or non-human primate, a mouse, a rat, a dog, a cat, a horse and/or a cow.

In the present application, the method or product of the present application are applicable to treatment of the nervous diseases or disorders. The nervous diseases or disorders may include degenerative diseases. The degenerative diseases are those with degeneration (for example, in terms of function, structure, and biochemistry) of a particular type of cell (for example, neuron), resulting in adverse clinical conditions. For example, Parkinson's disease is a degenerative disease occurred to the basal ganglia in the central nervous system. The degenerative diseases that can be treated using a homogeneous cell population of the present application include, for example, Parkinson's disease, multiple sclerosis, epilepsy, Huntington's disease, dysmyotonia (myodystonia) and choreoathetosis.

In another aspect, the present application provides a method for preventing, treating, or alleviating nervous system diseases or disorders (for example, Parkinson's disease), comprising administering the cell product and/or the pharmaceutical composition as defined to a subject in need thereof.

In another aspect, the present application provides use of the cell product and/or the pharmaceutical composition as defined in preparation of medicaments for prevention, treatment or alleviation of nervous system diseases or disorders (for example, Parkinson's disease).

In another aspect, the present application provides use of the cell product and/or the pharmaceutical composition as defined for prevention, treatment or alleviation of nervous system diseases or disorders (for example, Parkinson's disease).

In another aspect, the present application provides a method for improving the efficacy of a cell product containing dopaminergic neural precursor cells in a cell replacement therapy for nervous diseases or conditions (for example, Parkinson's disease) and enhancing the safety of transplantation.

In the present application, a rotation test may be used to verify the therapeutic effect of the cell product containing the dopaminergic neural precursor cells for the neurological disorders or disorders (for example, Parkinson's disease). For example, under certain conditions, the smaller the number of rotations in the rotation test, the better the therapeutic effect can be shown.

In the present application, the neural precursor cells having one or more of the features of CLSTN2⁺, PTPRO⁺, NTRK3⁺, FLRT2⁺, KITLG⁺, CD83⁺, and/or LMX1A⁺EN1⁺, can alleviate the symptoms of Parkinson's disease, and/or can improve the safety of the cell replacement therapy, and/or can improve the efficacy of the cell replacement therapy.

The cell product and/or pharmaceutical composition of the present application may be, for example, transplanted or placed in the central nervous system (for example, the brain or spinal cord), or the peripheral nervous system. The transplantation location in the nervous system for the cell product/or pharmaceutical composition as defined is determined based on a specific neurological condition. For example, the cell product/or pharmaceutical composition may be directly injection to the injured striatum, spinal parenchyma or dorsal ganglia. For example, the cell product and/or pharmaceutical composition of the present application may be transplanted into or near the striatum of a patient suffering from Parkinson's disease. Depending on the location of the neurological condition and the medical condition of the patient, those skilled in the art will be able to determine the most appropriate method or cell transplantation. The cell product and/or pharmaceutical composition of the present application can be co-administered with other therapies for treatment of the nervous diseases or disorders.

The present application further provides grafts (for example, graft compositions) after in vivo or in vitro differentiation, and use of these grafts or graft compositions.

Not wishing to be bound by any particular theory, the following examples are merely to illustrate the cell products, preparation methods, uses or the like according to the present application, and are not intended to limit the scope of the invention of the present application.

### Examples

### Experimental Models and Experimental Subjects

### 1. Cell Culture

H9 human embryonic stem cell (hESC) lines, induced pluripotent stem cells (hiPSCs) derived from blood cells derived from normal humans, hESC reporter cell lines, and hiPSC reporter cell lines were cultured on paved mouse embryonic fibroblasts (MEFs) with growth inhibited by irradiation. A culture solution consisted of DMEM/F-12, 1×NEAA, 0.5× Glutamax, 0.1 mM 2-mercaptoethanol, and 4 ng/ml FGF-2. The fresh culture solution was changed once a day, and passaging was conducted with Dispase II weekly. In some experiments, hESCs/hiPSCs were cultured under trophoblast-free conditions. Specifically, the cells were cultured on vitronectin; mTeSR™ Plus used as the culture solution was replaced every 2 days; and passaging was conducted with TrypLE™ Express Enzyme (1×) every 5 days.

### 2. PD Model and Cell Transplantation

All animal experiments were conducted according to protocols approved by the Center for Excellence in Brain Science and Intelligence Technology of the Chinese Academy of Sciences. The surgical procedures for constructing PD models in SCID mice have been described in previous studies (Chen et al., 2016). The adult SCID mice (8-12 weeks) were anesthetized with the mixture of 1%-2% isoflurane and oxygen. By means of the brain stereotaxic technique, 1 μL of 6-OHDA (3 mg/ml, dissolved in normal saline containing 1% ascorbic acid) was injected directly into the left substantia nigra of the brain (anteroposterior [AP]=−2.9 mm, lateral [L]=−1.1 mm, vertical [V]=4.5 mm, and vertical depth calculated from the skull). 4 weeks after the surgery for 6-OHDA injury, amphetamine-induced animals capable of rotating at 5 turns/min within 1 h were selected for cell transplantation. The animals were randomly grouped, and dopaminergic neural precursor cells or an equal volume of artificial cerebrospinal fluid (ACSF) (control) was transplanted. 50,000 cells were resuspended in 1 μL of ACSF containing a Rock inhibitor (0.5 μM), B27, and 20 ng/ml BDNF, and then injected into the left substantia nigra (anteroposterior [AP]=−2.9 mm, lateral [L]=1.1 mm, vertical [V]=4.4 mm, and vertical depth calculated from the skull) or the left corpus striatum (AP=+0.6 mm, L=1.8 mm, V=3.2 mm, and vertical depth calculated from the dura mater).

### Experimental Method

1. CRISPR/Cas9-Mediated Gene Editing and Generation of hESC Reporter Cell Line

To establish the reporter cell line, target position-targeted guide RNAs were designed by Web tools (https://zlab.bio/guide-design-resources and https://www.benchling.com/). Donor plasmids were designed according to the following structure: a 5⁺ homologous arm included a genome sequence of about 1,000 bp before the stop codon of a selected gene; P2A and tdTomato were inserted into selected genes (LMX1A, CLSTN2, and PTPRO) behind the stop codon; human GH poly A, a mouse PGK promoter, a puromycin resistance gene and a poly A sequence were inserted behind the P2A and tdTomato; and a 3⁺ homologous arm consisted of a stop codon and a genome sequence of about 1,000 bp thereafter.

For the LMX1A-tdTomato/EN1-mNeonGreen reporter gene line, the LMX1A-tdTomato hESC cell line was generated first, and then P2A and mNeonGreen were inserted into an EN1 donor plasmid with the same structure as LMX1A-tdTomato but using the neomycin resistance gene.

To establish surface marker reporter gene/EGFPnls cell lines for scRNA-seq, AAVS1-NeoR-CAG-EGFPnls-WPRE-poly A donor plasmids were constructed and electroporated into H9 ES cells using transcriptional activated effector nucleases (TALENs), or electroporated into well-constructed surface marker reporter gene cell lines.

For the TH-tdTomato/Surface Marker-EGFP reporter lines, they were further constructed based on the TH-tdTomato cell lines (for the construction of the cell lines, see Xiong, M. et al. (2021). Human Stem Cell-Derived Neurons Repair Circuits and Restore Neural Function. Cell Stem Cell 28, 112-126.e6.). Briefly, by replacement of tdTomato with EGFP, surface marker-tdTomato donor plasmids were reconstructed using the neomycin resistance gene, and the targeted guide RNAs were the same as that used for the surface marker-tdTomato cell lines.

Details of electroporation, genomic DNA extraction, and genomic PCR identification have been described in previous reports (Chen et al., 2015, 2016; Xiong et al., 2021).

### 2. Differentiation of Midbrain Central Neural Cells

The method for induction of midbrain ventral neural cells containing midbrain dopaminergic neural cells is developed by making modifications based on the method established previously (Xi et al, 2012). Briefly, the human embryonic stem cells or human induced pluripotent stem cells (1 day after passaging) were cultured in a neural induction medium (NIM) supplemented with SB431542 (10 μM) and DMH-1 (2 μM). To produce midbrain floor plate precursor cells from differentiated cells, SHH and CHIR99021 were added to the culture from Day 1 to Day 7. On Day 7, the neuroepithelial cell colony was gently blown down with a pipette, and then subjected to monolayer culture again for 6 days (D7-D12) with the NIM supplemented with SHH and CHIR99021. On day 12, the CHIR99021 was removed; the concentration of SHH was reduced to a certain concentration; and SAG and FGF8b were added to the culture solution to amplify the precursor cells in the suspension until Day 19. On Day 21, the precursor cells were cultured in the neural induction medium containing SHH and FGF8b until transplantation. After Day 36, the culture was continued with a neural differentiation medium (Neurobasal medium, 1×N2 supplement, 1×B27) (NDM) supplemented with a brain-derived neurotrophic factor (BDNF), a glial cell line-derived neurotrophic factor (GDNF), an ascorbic acid (AA), CAMP, and a transforming growth factor β3 (TGFβ3).

### 3. Cell Sorting and Flow Cytometry Assay

At room temperature, neurospheres were treated with Accutase for 8 min to dissociate them into single cells. The sorted cells were reseeded on a 96-well plate (Lipidure-Coatded Plate A-96U) with low cell adhesion at a density of 10,000 cells/well. The analysis was performed on the BD LSRFortessa flow cytometer (BD, USA), and the data were further analyzed using FlowJo software. A Rho kinase (ROCK) inhibitor (0.5 μM, Tocris) and a 10% vitamin A-free B-27 supplement were added to improve cell viability after seeding.

For cell lines sorted on Day 28, the culture was continued with the neural differentiation medium (Neurobasal medium, 1×N2 supplement, 1×B27) (NDM) supplemented with the brain-derived neurotrophic factor (BDNF, 10 ng/ml), the glial cell line-derived neurotrophic factor (GDNF, 10 ng/ml), the ascorbic acid (AA, 200 μM), cAMP (1 μM), and a transforming growth factor β3 (TGFβ3, 1 ng/ml).

For cell lines sorted on Day 21, neurospheres were cultured in the neural induction medium (NIM) containing 20 ng/ml SHH, 20 ng/ml FGF8b, and 50 mg/ml penicillin/streptomycin. Till Day 30, neuron maturation were then conducted, i.e., continuing the culture with the neural differentiation medium (Neurobasal medium, 1×N2 supplement, 1×B27) (NDM) supplemented with the brain-derived neurotrophic factor (BDNF, 10 ng/ml), the glial cell line-derived neurotrophic factor (GDNF, 10 ng/ml), the ascorbic acid (AA, 200 μM), CAMP (1 μM), a transforming growth factor β3 (TGFβ3, 1 ng/ml), and 50 mg/ml penicillin/streptomycin.

4. The 10× Chromium Platform scRNA-Seq was Used.

For in vitro differentiated samples, the attached cell clones (Day 8) or neurospheres (Days 14, 21, 28, and 35) were digested with TrypLE Express (ThermoFisher) for 10 min at 37° C. and washed twice with MIM. Then, the cells were sieved through a 35 μm cell sieve (BD) to obtain a single-cell suspension. According to the manufacturer's recommendations (10× Genomics), a library was prepared using a chromium single-cell 3⁺ kit (v2) or (v3). The library was sequenced on Illumina Hiseq PE150.

For transplantation samples, mDA neural precursor cells derived from the surface marker-tdTomato/AAVS1-CAG-EGFPnls-WPRE-polyA cell line or AAVS1-CAG-EGFPnls-WPRE-polyA cell line were transplanted into the striatum of PD mice of 4 months old, and the PD mice were sacrificed with an excessive dose of chloral hydrate, followed by transcranial perfusion with cold and oxygenated artificial cerebrospinal fluid (aCSF, in mM: 124 NaCl, 2.5 KCl, 1 NaH₂PO₄, 25 NaHCO₃, 37 glucose, 2 CaCl₂), 2 MgSO₄). The brain was removed; 200 μm of the brain were collected and sectioned over vibration; and graft regions (mostly located in the striatum) were microdissected under a stereofluorescence microscope with a cooling plate. These brain slices were treated with a self-made ice-cold oxygenated dissection medium (DM, in mM: in mM: 81.76 Na₂SO₄, 120 K2SO₄, 5.8 MgCl₂, 25.2 CaCl₂), 1 HEPES, 20 glucose, 20 NaOH) supplemented with papain (20 units/ml, Worthington), 0.067 mM 2-mercaptoethanol (Sigma), 1.1 mM EDTA, cysteine, and DNase I, and then enzymatically digested for 30-40 min, followed by manual pipetting using a fire-polished Pasteur pipette and filtration through a 35 μm DM-equilibrated cell strainer (BD). Then, the cells were pelleted at 400 g for 5 min, the supernatant was carefully removed, and then the cells were resuspended in 1-2 ml of DM containing 2.5% FBS. Then, according to the manufacturer's recommendations (Miltenyi Biotec), the debris removal step was conducted on the cell suspension using a debris removal solution. The cell pellets were then resuspended in 200-400 ul of DM containing 2.5% FBS for cell sorting, in order to enrich EGFP-positive human grafts. According to the manufacturer's recommendations (10× Genomics), a library was prepared using a chromium single-cell 3⁺ kit (v3). The library was sequenced on Illumina Novaseq 6,000.

5. Preprocessing of scRNA-Seq Data

The scRNA-seq data were aligned to the human reference genome GRCh38-3.0.0, and demultiplexed using the default parameters of the Cellranger software (10× Genomics, v3.0.2 or v4.0.0). The obtained filtered count matrix was used for downstream analysis.

### 6. Clustering and Identification of Cell Population

The filtered count matrix was analyzed and processed using Seurat and Scanpy, in terms of data filtering, normalization, highly variable gene selection, scaling, dimensionality reduction, and clustering. First, the scRNA-seq sampled at each time point was created separately as a Seurat object. Genes with less than 3 counts were removed, and cells with fewer than 200 genes detected were removed. Next, each Seurat object was converted to a loom file and imported into Scanpy for clustering. Then, six Seurat objects were merged using the “Merge” function in Seurat and converted to a loom file for cell type clustering. The details of downstream analysis were as follows.
6.1. Data filtering: Cells with a mitochondrial gene proportion greater than 5% were excluded. Then, cells with more than 1,000 genes detected, cells with fewer than 6,000 genes detected (cells with 6,000 genes detected had potential doublets), and cells with more than 1,000 counts detected and fewer than 40,000 counts detected (cells with more than 40,000 genes detected were potential amphicytes) were retained.
6.2. Data normalization: For each cell, logarithmic normalization was conducted using the “NormalizeData” function in Seurat, with “scale.factor” set to 40,000.
6.3. Selection of highly variable genes: 2,000 highly variable genes were calculated using the “FindVariableFeatures” function in Seurat. We then identified genes associated with the cell cycle marker TOP2A (with the Pearson correlation coefficient greater than 0.15), and excluded them from 2,000 highly variable genes.
6.4. Cell cycle scoring: With the “CellCycleScoring” function, in a cell cycle-related gene set, 43 genes expressed in a G1/S stage and 54 genes expressed in the G2/M stage were used to calculate the S-stage score and the G2M-stage score. The cell cycle difference score was the difference between the S-stage fraction and the G2M-stage fraction.
6.5. Data scaling: The “ScaleData” function was performed for the Seurat objects using default parameters. The number of counts, the number of genes, the proportion of mitochondrial genes, and the cell cycle difference score were variables regressed in “ScaleData”.
6.6. Principal component analysis: Highly variable genes were used to calculate principal components in the “RunPCA” function. 100 principal components (PCs) were obtained and stored in Seurat objects to calculate neighborhood graphs and umaps in the next section.
6.7. Leiden clustering: The Seurat objects were converted to loom files and imported by Scanpy. The neighborhood graphs of the observed values were calculated by “pp.neighbors” of Scanpy. Then, the Leiden algorithm was used by “scanpy.tl.leiden” to calculate cell clustering.
6.8. Cell population merging and pruning: The top 200 differentially expressed genes in each cell population passed “scanpy.tl.rank_genes_groups”. Cell population annotation was done manually based on previously classical markers in the developing midbrains and the mouse developmental brain atlases. For cell populations having similar marker gene expression, we combined them into a single population. For cell populations having unknown marker gene expression, we defined their cell population identities using the web-based gene annotation analysis tool Metascape (Zhou et al., 2019). In conjunction with the filter count matrix and the Metascape's analysis results, we defined certain cell populations that were low-quality (with a small number of cells and genes detected), stressed, and apoptotic with high detection rate of ribosomal protein genes and low oxygen response. We then filtered them out to obtain the final pruned list of cells. Finally, we conducted analysis steps 1-8 again to obtain consistent cluster annotations in separate point-in-time datasets and a combined dataset.

### 7. Regional Gene Module Score Analysis

Based on the detected genes in our dataset, previous studies and mouse developmental brain atlases, regional gene modules were collated. Briefly, a midbrain gene module included OTX1, OTX2, LMX1A, EN1, PITX2, and SIM2. A hindbrain gene module included HOXB-ASI, HOTAIRMI, HOXA2, HOXB2, GATA3, and GBX2; and an MHB gene module included FGF8, FGF17, NKX2-8, and PAX8. Then, the scores of the gene modules were calculated with “scanpy.tl.score_genes”. Regional classification was defined based on the expression of three gene modules in UMAP embedding.

### 8. Single-Cell RNA Velocity Analysis

First, the command line interface of velocyto was applied to generate spliced/unspliced expression matrices for the scRNA-seq data at each time point (La Manno et al., 2018). All matrices were then merged in a loom form and subjected to downstream RNA velocity analysis using scVelo (Bergen et al., 2020). 21,531 genes with fewer than 20 counts were screened, the top 2,000 highly variable genes were retained, and all cells used for clustering in the combined time-course dataset were intersected. The complete splicing kinetics of 2,000 highly variable genes was detected, the velocity was estimated in a “random” mode, and a velocity graph was calculated based on cosine similarity. A velocity flow graph was plotted on the umap embedding. Based on velocity estimates in the “random” mode, a generic gene-sharing latency time that represents the cell's internal clock was calculated based only on the transcriptional kinetics of the cells, and plotted on the Umap embedding.

### 9. Pseudotime and Gene-Level Cascade Analysis

Only mDA-related cell populations (0-P_MesenFP_LMX1A_Early, 1-P_MesenFP_LMX1A_Late, 2-P_MesenFP_D14, 11-N_DA, and 14-N_DA_Neuroblast) were extracted and used for pseudotime analysis. 1978 genes detected in less than 3 cells were filtered out. Then, the clustering and identification of similar cell populations in steps 2-6 were conducted, including data normalization, selection of highly variable genes, cell cycle scoring, data scaling, and principal component analysis. The pseudotime value was calculated using the URD software package (Farrell et al., 2018). Briefly, the diffusion diagram was calculated using the “calcDM” function in URD. The P_MesenFP_D14 cell population was selected as root cells, and the pseudotime was calculated by performing a probability breadth-first search for the kth nearest neighbor graph. To find genes that vary with pseudo-time, we considered only genes expressed in at least 1% of cells as “expressed genes.” We then calculated a spline curve that is applicable to the average expression of five cells per group. We selected genes that change over time, where: (1) the actual average expression value changed by at least 0.5, and the average expression value of log 2 changed by at least 20%; (2) the spline curve fits well, and here we set the threshold value of 0.045 for the residual sum of squares: and (3) the fit of the spline curve was significantly better than a flat line with a slope of 0, and here we set the threshold value of 0.25 to be better than flat. Next, we used the intersection of these genes for the gene expression cascade.

### 10. Cell Type Reproducibility Analysis

To evaluate the similarity between human midbrain-generated cell types developed in vitro and in vivo, the MetaNeighbor R software package (v1.6.0) was used to calculate AUROC scores, which were taken as performance carriers for neuronal types and neural precursor cell types, respectively. First, we selected 2,000 genes as integration features for the Seurat object and prepared a public dataset into the Seurat object. Then, anchors found using the ‘FindIntegrationAnchors’ function integrated the two datasets. Next, with the SummarizedExperiment R package (v1.16.1), we prepared a normalized data matrix of 2,000 anchor genes (variable genes) as the SummarizedExperiment class. The fast, low-memory, and unsupervised version of MetaNeighbor was used to calculate AUROC scores (MetaNeighborUS function, the fast version was used). The average AUROC score across datasets was plotted in a heat map.

11. Integration of Graft scRNA-Seq Datasets

For the integrated analysis of the scRNA-seq datasets of transplantation samples, Harmony integration was used to reduce the technical batch effect between different marker-sorting groups (two batches for the unclassified group, one batch for the CLSTN2-derived group, and one batch for the PTPRO-derived group). The “RunHarmony” function in the Harmony software package was used to calculate corrected Harmony coordinates. Then, the Seurat object was converted to a loom file and imported by Scanpy. The neighborhood graphs of the observed values were calculated by “scanpy.pp.neighbors”. Then, the cells were clustered using the leiden algorithm by means of the “scanpy.tl.leiden” function in Scanpy, and corrected Harmony embedding instead of a PC was used for this function. Cell population annotation was done manually based on classical markers of previous major cell types. For further clustering among neurons, neurons were filtered by means of STMN2 expression and then clustered, similar data processing was performed repeatedly, and the clustering and identification of similar cell populations was performed in steps 2-6.

### 12. Estimation of Proportions of Certain Gene-Positive Cells Across Stages

To estimate the percentage of a cell population that certain genes represent, 10% of cells were randomly selected from the scRNA-seq dataset at each time point, and the process was repeated 10 times as 10 trials. We then set a threshold (UMI number>0) for the UMI count of certain genes. Finally, we designated cells whose specific genes exceeded the UMI count threshold as positive cells.

For average pseudo-batch expression, the gene expression was averaged by stage of the combined timecourse scRNA-seq dataset by using “Average Expression” in Seurat. The average expression levels of the selected genes (LMX1A, EN1, CLSTN2, and PTPRO) were extracted and plotted.

### 13. Transcriptome Sequencing and Data Analysis

For the transcriptional analysis of the LMX1A-tdTomato/EN1-mNeonGreen reporter gene line, the LMX1A-tdTomato/EN1-mNeonGreen cell line was directionally differentiated towards dopaminergic neurons. The cells were digested as described above and sorted with the BD LSRFortessa flow cytometer on Day 21 or 28. tdTomato+Neongreen+ was collected as one group, while tdTomato-Neongreen+, tdTomato+Neongreen− and tdTomato−Neongreen− were collected together as another group. The cells were precipitated under 400 g within 5 min and lysed with TRIzol. The sequencing library was prepared using NEBNext® Ultra™ RNA Library Prep Kit for Illumina®) (NEB, USA) according to the instructions of sample manufacturer. The library was sequenced on Illumina Hiseq PE150.

The reading of the original sequencing sequence was processed by quality control and adaptor modification. Then, a clean sequencing read was mapped to the UCSC human GRCh38 genome using HISAT2 software (version 2.1.0). The bam file was generated using SamTools (version 0.1.19). The reading was counted by the “summarizeOverlaps” of the GenomicFeatures package. Differential expression analysis was conducted using the ‘glmTreat’ function in the edgeR software package. DEGs were selected according to the following criteria: the absolute fold change was greater than 1.5 (| Log 2FC|>1.5), and the Benjamini-Hochberg corrected p-value of the cut-off value was 0.05.

For the DEG volcano map, a statistically significant critical value of 0.001 was chosen, and a critical value of 2 was chosen for the absolute log 2 fold change. The volcano map was implemented using the Enhanced Volcano R software package (v1.4.0).

### 14. Estimation of Cell Type Proportion in Transcriptome Sequencing Using Single-Cell Data

For deconvolution analysis, with Music (Wang et al., 2019), the cell type proportion was estimated for the transcriptome data using the scRNA-seq data from the corresponding stage (Days 21 and 28). With the Biobase package, both the transcriptome dataset and the scRNA-seq dataset were prepared into “ExpressionSet” objects. By means of the default single-tail Wilcoxon rank sum test, the input gene markers for each scRNA-seq cell type were extracted from the top 500 DEGs calculated by the “FindAllMarkers” function.

### 15. Tissue Preparation and Immunohistochemistry

Animals were sacrificed with an excess of pentobarbital (250 mg/kg, intraperitoneal) and perfused first with saline followed by paraformaldehyde (PFA) buffered with 4% ice-cold phosphate. The brains were removed and then immersed in 20% and 30% sucrose until submergence. On a freezing microtome (Leica SM2010R), the brains were cut by the thickness of 30 mm to obtain a sequence of sagittal (0.12 mm to 3.12 mm from inner side to outer side) or coronal (1.42 mm to 0.10 mm from bregma) slices, which were stored in a cryoprotectant solution at 20° C. The floating brain slices were incubated with the primary antibodies for 1-2 nights at 4° C., and then the unbound primary antibodies were removed. For DAB staining, the slices were incubated with the corresponding biotinylated secondary antibodies for 1 h, and then incubated with avidin-biotin peroxidase for 1 h at room temperature. The immunoreactivity was observed using a DAB staining kit. The slices were then dehydrated with ethanol, permeabilized in xylene, and immobilized in neutral resin. For fluorescent immunolabeling, the slices were incubated with the corresponding fluorescent secondary antibodies for 1 h at room temperature. The slices were then sealed by means of Fluoromount-G.

### 16. Imaging and Cell Quantification

To quantify the number or proportion of TH cells expressing EN1, FOXA2, LMX1A, and GIRK2 in the TH cells, at least 5 images randomly selected from coverslips were counted using ImageJ software. The data were repeated three times and expressed as mean±SEM. To measure the human fiber density in brain slices, tiled images were captured using a Nikon TE600 or Olympus VS120 microscope. The optical density of the human brain in different regions of the mouse brain was measured by an image processing and analysis system (Image Pro Plus 5.1 software). The data were displayed as optical density in different regions. For TH, GIRK2, LMX1A, human nuclei (hN), and FOXA2 staining, grafts were outlined and captured using a Nikon TIE inverted laser scanning confocal microscope (Nikon, 60× objective lens) or Olympus VS120 (Olympus, 20× objective lens). Single- or double-stained cells were manually counted with ImageJ. Data were expressed as the proportions of TH−, LMX1A−, FOXA2− and total hN, or the proportions of GIRK2/TH/hN and TH/hN cells. All data were expressed as mean±SEM.

To estimate the graft volume, the fraction immunostained with hN was magnified 20-fold and analyzed with ImageJ. The transplantation area was extrapolated by each part of 1:6, and the volume was calculated based on the Cavalireis principle. To quantify the average gray values of tdT+ fibers, all conditions including staining and consistent capturing. Four regions on the left and right sides of the striatum were selected, the threshold was set based on the Li algorithm, and the pixels within the threshold were quantized. The relative average gray value was defined as the average gray value of the transplanted site minus the average gray value of the non-transplanted site.

### 17. Behavioral Test

### 17.1. Rotation Test

Amphetamine-induced rotation was tested before transplantation and each month till the sixth month after transplantation. 5-10 min after peritoneal injection of amphetamine (in 2 mg/ml normal saline, 5 mg/kg), camera recording was conducted for 2 h. Data were expressed as the average net revolutions per minute over 90 minutes. The video was analyzed manually. Both ipsilateral and contralateral rotations were calculated. The data shown included net ipsilateral rotations over 60 minutes. Animals exhibiting behavioral defects (i.e., greater than 300 rotations over 60 min) were defined as successful PD models and were available cell transplantation. Rotation test was conducted on Months 2, 4, and 6 after transplantation.

### 17.2. Quantitative and Statistical Analysis

Statistical analysis was conducted using SPSS software. In all studies, data analysis was performed by Student-t-test, paired t-test, two-way ANOVA, Holm-Sidak test, Two-way ANOVA followed by Holm-Sidak test and Tukey's post hoc test or one-way ANOVA and Holm-Sidak test. Statistical significance was determined as p<0.05. *, **, and *** represent p<0.05, p<0.01, and p<0.001, respectively.

### 18. Whole-Cell Patch-Clamp Recording of Brain Slices

5 months after cell transplantation, horizontal coronal brain slices (300 mm thick) of the forebrain were prepared from PD mice using a vibratome (Leica VT1200S) in an ice-cold cutting solution (in mM: 100 glucose, 75 NaCl, 26 NaHCO₃, 2.5 KCl, 2MgCl₂₋6H₂O, 1.25 NaH₂PO₄-6H₂O and 0.7 CaCl₂)). The slices were transferred into an artificial cerebrospinal fluid (aCSF, in mM: 124 NaCl, 4.4 KCl, 2 CaCl₂), 1 MgSO₄, 25 NaHCO₃, 1 NaH₂PO₄, and 10 glucose) under a saturation condition with 95% 02 and 5% CO2, treated for 12 min at 32ºC and then transferred to aCSF at room temperature. After 60 min of recovery, the slices were transferred to a recording chamber, into which oxygenated aCSF was continuously perfused at 28° C. at a rate of 2-4 ml/min. The transplanted mDA neurons were identified by tdT fluorescence in the grafts.

The initial access resistance in a range of 15-30 M2 was monitored throughout the experiment. Cells with access resistance change greater than 15% were discarded. Data was filtered at 1 KHz and digitized at 10 kHz. Voltage and current signals were recorded using the Axon 700B amplifier (Axon). The recording electrode (3-5 M(2) filled with an internal solution (in mM: 120 K⁺-glucose, 5 NaCl, 0.2 EGTA, 10 HEPES, 2 MgATP, 0.1 Na₃GTP, and 10 phosphocreatine, with the pH adjusted to 7.2 with HCl) was used for action potential recording. The action potential (AP) in response to the depolarization current (0-180 pA, 20 pA step size, for 600 ms) was recorded in a current clamp mode. Ramp current injections (100-300 pA, for 2,000 ms) were used to record the maximum discharge frequency of mDA neurons. In the current clamp mode, voltage dip measurements were conducted on the transplanted mDA neurons by injecting current (−120 pA, for 2,000 ms).

The recording electrode (3-5 M(2) filled with an internal solution (in mM: 112 Cs-gluconate, 5 TEA-CI, 3.7 NaCl, 0.2 EGTA, 10 HEPES, 2 MgATP, 0.3 Na₃GTP, and 5 QX-314, with pH adjusted to 7.2 with CsOH) was used to record spontaneous excitatory postsynaptic currents (sEPSCs) and spontaneous inhibitory postsynaptic currents (sIPSCs). To record the sEPSCs and sIPSCs, the voltages for cells were −60 mV or −10 mV, respectively.

### Example 1 Discovery of Novel Molecular Markers for mDA Precursor Cells

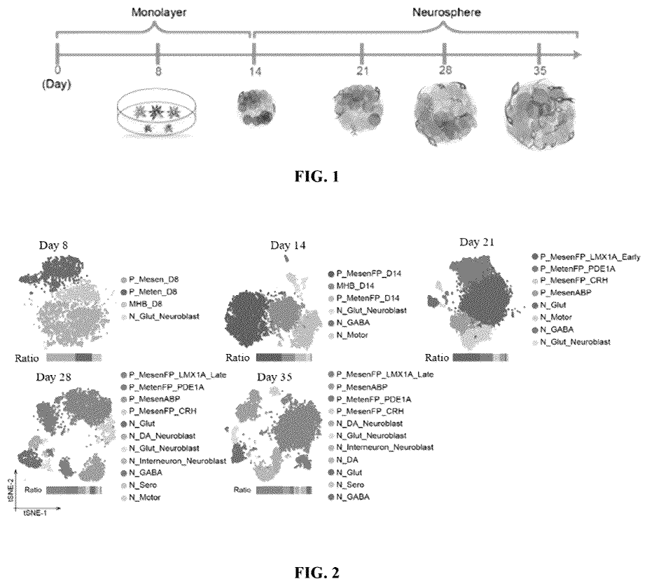

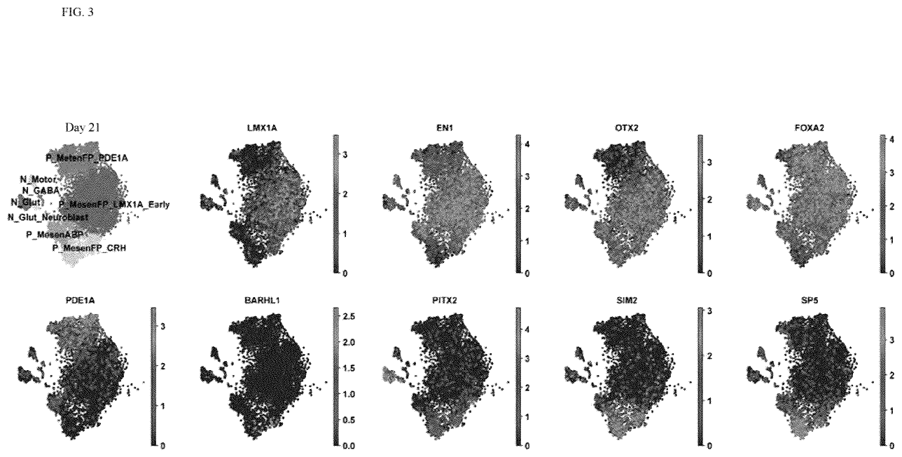

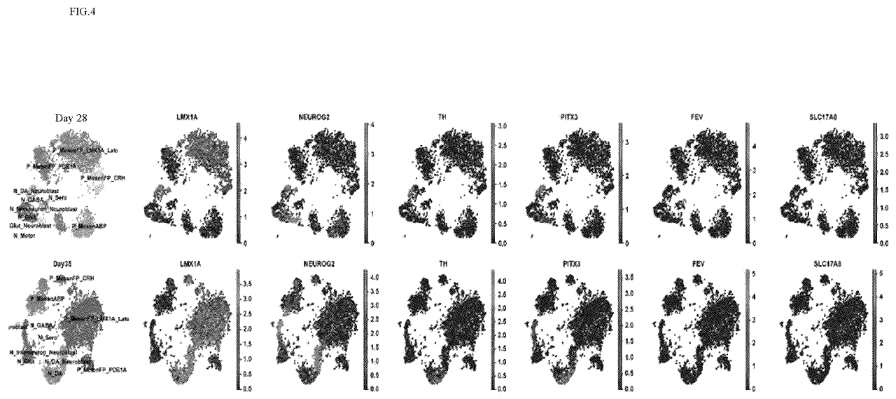

The inventors of the present application analyzed the differentiation process and heterogeneity of hPSCs-derived midbrain ventral neural cells by using high-throughput single-cell transcriptomics. Specifically, the inventors simulated the development of neurons in vivo, and used an improved protocol to directionally differentiate the hPSCs into midbrain ventral neural cells including mDA neural cells (Xi et al., 2012; Xiong et al., 2021) (FIG. 1). The mDA precursor cell population was initially detected on Day 21 (cell population P_Mesen_LMX1A_Early, Day 21, FIG. 2), and was featured with co-expression of typical midbrain ventral floor plate neural precursor cell markers (LMX1A⁺, EN1⁺, OTX2⁺, and FOXA2⁺, FIG. 4). Other neural precursor cell populations were also detected, including a group of hindbrain floor plate neural precursor cells (cell population P_MetenFP_PDEIA, Day 21, PDE1A⁺, EN1⁺ and OTX2⁻, FIGS. 2 and 3), and two groups of midbrain floor plate neural precursor cells (cell population P_MesenFP_CRH, BARHL1⁺ and PITX2⁺; cell population P_MesenFP_ABP, SIM2⁺ and SP5⁺, FIGS. 2 and 3). Till mid-to-late differentiation, on Days 28 and 35, mDA neuroblasts (LMX1A and NEUROG2⁺), mDA neurons (TH⁺ and PITX3⁺), and a fraction of 5-hydroxytryptaminergic neurons (FEV⁺ and SLC17A8⁺) were observed (FIGS. 2 and 4).

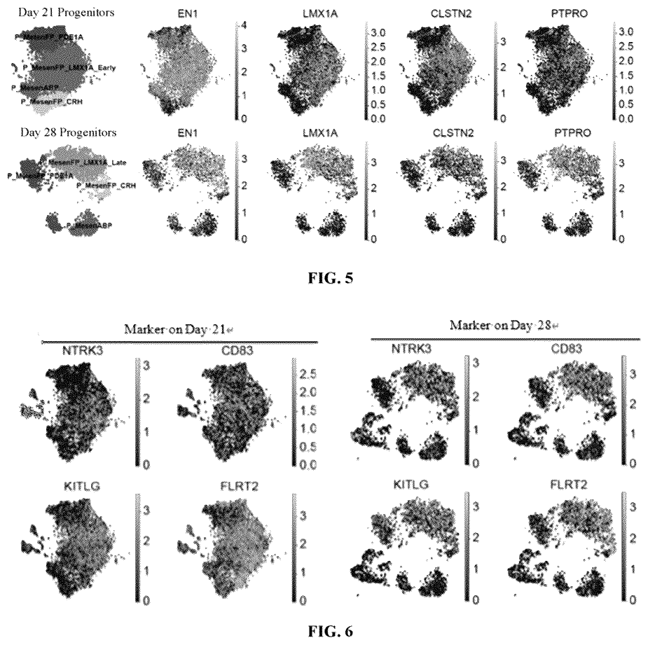

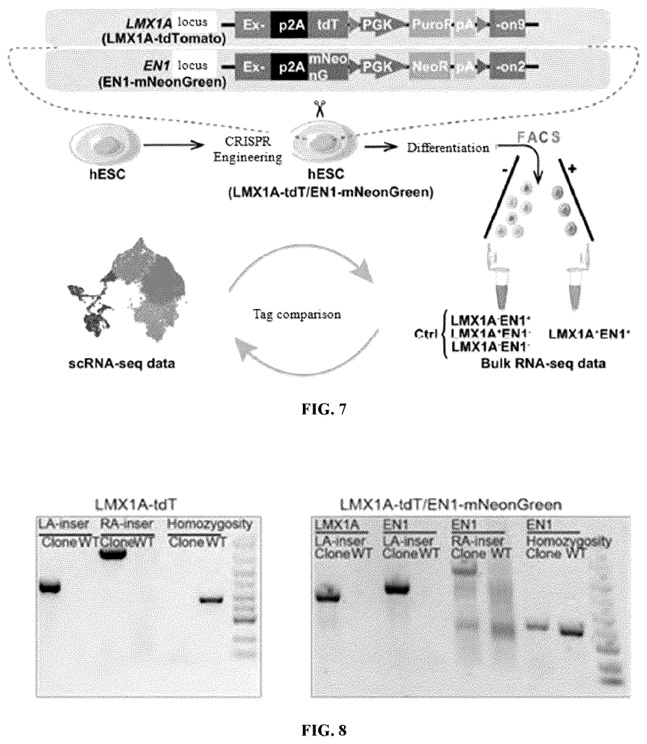

Surprisingly, the inventors discovered novel molecular markers CLSTN2, PTPRO, NTRK3, FLRT2, KITLG, and CD83 specifically expressed on mDA precursor cell populations, and found that the combination of classical transcription factors LMX1A and EN1 in midbrain ventral floor plate neural precursor cells could specifically represent mDA precursor cell populations (FIGS. 5 and 6).

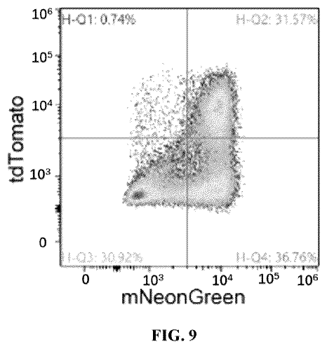

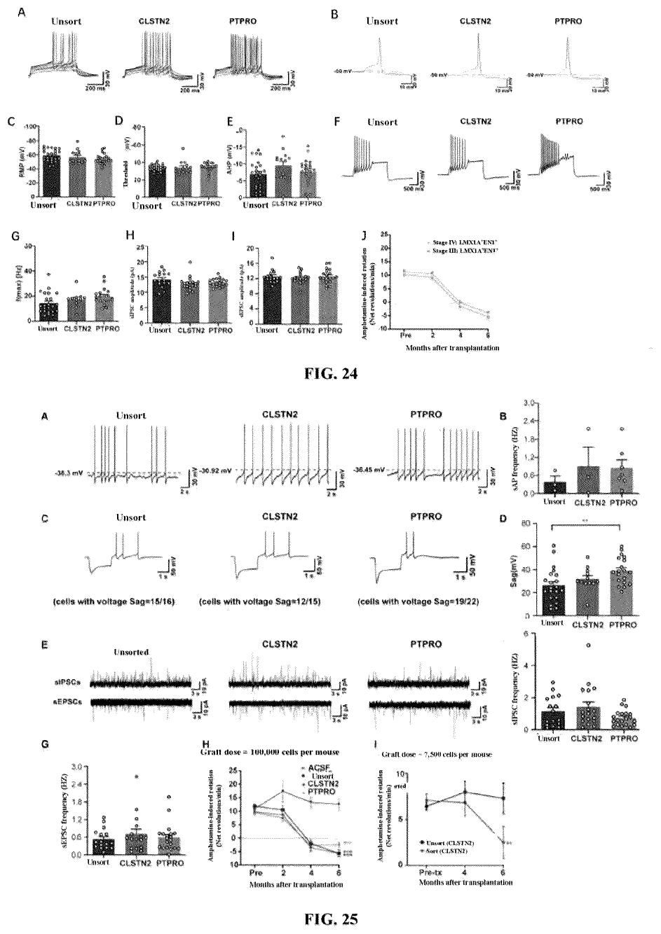

To further verify the expression of these novel molecular markers in the mDA neural precursor cells, the inventors analyzed the assumed differentially expressed genes between the mDA precursor cells and other cells by batch transcriptome sequencing. The inventors first constructed a dual fluorescent reporter cell line by inserting two fluorescent proteins into the LMX1A and EN1 genetic loci by a CRIPSR/Cas9 technique, respectively (FIGS. 7 and 8). On Days 21 and 28, the inventors isolated double-positive cells by a flow cytometry fluorescence sorting (FACS) technique (FIGS. 7 and 9). Other cells (single-positive cells and double-negative cells) served as control cells (FIGS. 7 and 9). According to batch transcriptomic analysis, CLSTN2 and PTPRO showed high expression in the double-positive group but low expression in control cells (FIG. 10, genes outlined in black, and left and middle panels in FIG. 11).

In addition, symbolic genes of other neuronal types or neural precursor cell types were highly expressed in the control cell group, indicating that cell types other than mDA precursor cells were excluded from the cell population represented by the double-positive marker (LMX1A⁺EN1⁺) (FIG. 10, genes not outlined). The inventors examined the top-ranked differentially expressed genes (DEGs) from batch transcriptome sequencing from the scRNA-seq datasets on Days 21 and 28, respectively. It could be clearly noted that the highly expressed genes in the double-positive group were specifically expressed on single-cell sequencing-annotated mDA precursor cell populations (FIG. 12A, star-labeled column), while other types of cell markers were distributed in other non-mDA precursor cell populations (FIG. 12A). The inventors further integrated two sets of data (batch transcriptome sequencing and single-cell sequencing), and estimated the cell type proportion for the batch transcriptome sequencing by using MuSiC (method). The heat map of proportion showed that the double-positive group subjected to batch transcription sequencing consisted of a high proportion of single-cell sequencing-annotated mDA precursor cell populations (P_MesenFP_LMX1A_Early and P_MesenFP_LMX1A_Late), while the cell transcription group of control cells consisted of a variety of non-mDA precursor cell population types (FIG. 12B).

To experimentally verify whether the two surface markers CLSTN2 and PTPRO were localized to the cell membrane, the inventors constructed two gene expression vectors, and inserted hemagglutinin tags (HA-tags) into the N-terminus (Construct I) or C-terminus (Construct II) of CLSTN2 or PTPRO, respectively (FIG. 13). The inventors transfected these plasmids separately into 293T cells. By immunostaining the HA-tags, the inventors found that the immune activity of HA-tags could be detected in both types of constructs in the cell fixation/membrane rupture state, while for the live-cell state (i.e., non-fixation/membrane rupture state), the immune activity of HA-tags was only detectable when the HA-tags were at the N-terminus. These experiments demonstrated that CLSTN2 and PTPRO were transmembrane proteins expressed on the cytoplasmic membrane.

### Example 2 Efficient Enrichment of mDA Neurons In Vitro by Sorting mDA Precursor Cells with Molecular Markers

In order to isolate cells rich in specific markers in a live cell state, this example constructed a fluorescent reporter cell line by knocking a fluorescent protein tdTomato into the C-terminus of each marker gene (CLSTN2 and PTPRO) by a CRISPR/Cas9 technique (FIGS. 14A, 14B, 15A). The inventors isolated tdTomato” cells at the stage of mass production of precursor cells (CLSTN2 on Day 21, PTPRO on Day 28), reaggregated single cells into neurospheres for culture, and switched to in vitro maturation using a neural differentiation culture solution from Day 30 (FIG. 15A). Prior to induced differentiation maturation (Day 30), the inventors detected the expression of the mDA precursor cell marker molecules LMX1A and OTX2 in sorted and unsorted neurospheres, and found that, compared with the non-sorting group, LMX1A or OTX2-positive cells enriched by CLSTN2, or PTPRO, or LMX1A⁺EN1⁺ double-positive sorting were significantly enriched in the neurospheres, suggesting enrichment of mDA precursor cells (FIG. 15B-15E). In the late stage of differentiation maturation (Day 45), by staining the dopaminergic neuron-specific marker TH, the inventors found that, compared to the non-sorting group, TH-positive neural cells enriched by CLSTN2, or PTPRO, or LMX1A⁺EN1⁺ double-positive sorting were significantly enriched in the neurospheres (the double-positive sorting group: 57.7%; the double-positive non-sorting group: 14.5%; the CLSTN2 sorting group: 46.3%; the CLSTN2 non-sorting group: 17.9%; the PTPRO sorting group: 41.7%; the PTPRO non-sorting group: 8.2%; FIGS. 15F-15G), demonstrating the enrichment of terminal dopaminergic neurons after sorting. In addition, the percentage of CLSTN2+ or PTPRO+ cells in unsorted neural precursor cells correlated well with the proportion of mDA neurons in mature neurospheres (for the CLSTN2 group, R=0.98, and P=0.0033; for the PTPRO group, R=0.94, and P=0.018; Pearson correlation) (FIG. 15H), indicating that the proportion of these two marker-positive cells in neural precursor cells was available for prediction of the yield of terminal mDA neurons.

The above neural precursor cells were differentiated from hESCs cultured in trophoblast cells. The inventors further detected the enrichment of CLSTN2 or PTPRO in neural precursor cells derived from differentiation of hiPSCs and in neural precursor cells derived from differentiation of hESCs cultured from trophoblast-free cells. Two hiPSCs derived from normal human blood cell were constructed, named XZ #2-hiPSCs and ZYW #2-hiPSCs, respectively. Further, the CRISRP-Cas9 technique was utilized to knock the tdTomato genes into the hiPSCs to obtain XZ #2-CLSTN2-tdTomato and ZYW #2-PTPRO-tdTomato reporter cell lines (FIG. 16A-16D). Compared with the non-sorting group, the neurospheres formed by HIPSCs-derived neural precursors enriched by CLSTN2 or PTPRO sorting were significantly enriched with TH-positive mDA neurons in the neurospheres after differentiation and maturation (the proportions of TH+ neurons in the XZ #2-CLSTN2 group: 50.2%±5.9% in the CLSTN2 sorting group, and 9.0%±0.9% in the non-sorting group. the proportions of TH+ neurons in the ZYW #2-PTPRO group: 41.6%±3.8% in the PTPRO sorting group and 13.2%±2.8% in the non-sorting group) (FIG. 16E-H). For the neural precursor cells derived by hESCs differentiation in a trophoblast culture condition, CLSTN2 sorting could significantly increase the proportion of TH-positive mDA neurons in mature neurospheres (the CLSTN2 sorting group, 44.0%±4.2%; and the non-sorting group: 7.7%±0.8%) (FIGS. 16H-16J). These results demonstrated that whether it was hESCs or hiPSCs, and whether trophoblast-cultured hESCs or trophoblast-free cultured hESCs, the neural precursor cells differentiated from these cells could achieve the final enrichment of dopaminergic neurons by means of CLSTN2 or PTPRO-mediated cell sorting.

### Example 3 Efficient Enrichment of mDA Neurons in Intracerebral Graft by Sorting mDA Precursor Cells with Molecular Markers

The inventors further detected whether intracerebral transplantation of neural precursor cells sorted by surface molecular markers could increase the proportion of mDA neurons within a graft block. In this example, the precursor cells sorted or unsorted using CLSTN2, or PTPRO, or LMX1A⁺EN1⁺ double-positive were transplanted into the striatum of the PD mouse models, respectively. Compared with the non-sorting group (9.80% of mDA neurons in the non-sorting group), it was found that the proportion of TH-positive mDA neurons in the sorting group with all conditions was significantly enriched (58.1% in the PTPRO group, 81.5% in the CLSTN2 group, 57.3% in the LLMXIA⁺ EN1⁺ group on Day 21, and 32.4% in the LMX1A⁺EN1⁺ group on Day 28; FIGS. 17A-17D), and these neurons all expressed the floor plate marker molecule FOXA2, further demonstrating that these TH-positive neurons were indeed midbrain floor plate-derived dopaminergic neurons (FIGS. 18A and 18B). The TH-positive neurons in the grafts co-expressed PITX3 and the dopamine transporter SLC6A3 (also known as DAT), demonstrating that these neurons were mature mDA neurons (FIGS. 18C and 18D).

By staining different subtypes of marker molecules of mDA neurons, the inventors further found that 90% of TH-positive neurons in the grafts co-expressed the marker molecule GIRK2 of the dopaminergic neurons (A9) in the substantia nigra of the midbrain, while less than 10% of TH-positive neurons expressed the marker molecule CB of the dopaminergic neurons (A10) in the ventral tegmental area of the midbrain, and there was no significant difference in the proportion of TH-positive neurons co-expressing GIRK2 or CB in the sorting group and the non-sorting group. It was suggested that the mDA neurons obtained after non-sorting and sorting enrichment were mainly dopaminergic neurons (A9) in the substantia nigra of the midbrain, and A9 was the subtype of mDA neurons mainly lost in PD (FIGS. 18E and 18F).

### Example 4 Analysis of Graft Composition by scRNA-Seq

The purpose of this example was to clarify the cell composition of the graft in the cell therapy. To facilitate the isolation of human-derived transplanted cells from the transplanted brains of PD mice, the inventors constructed hPSCs cell lines expressing nucleo-localized EGFP based on the fluorescent reporter cell lines of surface markers (FIG. 19A). After 4-5 months since transplantation of CLSTN2 or PTPRO-sorted or unsorted precursor cells into the striatum, the transplanted mouse brain was dissected and digested into a single-cell suspension to isolate human cells by FACS for single-cell sequencing (two batches of unclassified groups, one batch for each surface marker group: FIG. 19A). By low-resolution clustering analysis, it was found that the graft was mainly composed of four major cell types, including oligodendrocyte precursor cells (OPCs) or oligodendrocyte cells (OPC/Oligo cell population), astrocytes (Astro cell population), neurons (Neuron cell population), and vascular pia mater cells (VLMC cell population) (FIGS. 19C and 19B). Here, the VLMC cell population was also found in midbrain ventral cell grafts derived from previous hPSCs (Tiklová et al., 2020). Further analysis revealed that the VLMC cells could be divided into different subpopulations, and that the subpopulation composition of VLMC differed in grafts from the sorting and non-sorting groups of different markers (FIG. 21A). Histological staining verified the presence of GFAP-positive astrocytes, OLIGO2-positive oligodendrocyte precursor cells or oligodendrocyte cells, and COLIA1-positive VLMC cells in the graft (FIGS. 21C-21D). By further statistical analysis of histological staining, it was found that the proportion of astrocytes was consistent in the sorting and non-sorting groups, whereas the OPC/Oligo cell population was almost undetectable in the sorting group (FIGS. 21F and 21G).

Further clustering analysis of neurons yielded 12 neuronal subtype cell populations (FIG. 19E), all of which could be distinguished by representative markers (FIG. 19F). The inventors detected three subtypes (DA_0, DA_1, and DA_2) of mDA neurons expressing TH and PITX3, and found that the DA_0 cell population expressed the dopamine transporter SLC6A3, also known as DAT, which possibly indicated that this population included mature mDA neurons (FIGS. 19E and 19F). Histological validation further demonstrated the presence of both DAT-positive and TH-positive mDA neurons in both unsorted and CLSTN2- or PTPRO-sorted grafts (FIG. 20A). Meanwhile, 5 different subtypes of GABAergic neurons, 3 different subtypes of glutamate neurons, and 5-hydroxytryptamine neurons were also detected. These results further demonstrated the presence of multiple types of neurons in the graft (FIGS. 19D and 19E). To compare changes in neuronal composition between surface marker sorting groups and non-sorting groups, the inventors calculated the percentages of different neuronal subtypes for each group (FIG. 19F). Similar to histological detection, the proportion of mDA neurons in the graft was significantly increased in the CLSTN2 or PTPRO sorting group (FIGS. 19A and 19B). In the non-sorting group, there were five different types of GABAergic neurons, accounting for 40.55% of the total number of transplanted neurons, and there is also one type of 5-hydroxytryptamine neurons, accounting for 5.78% of the total number of transplanted neurons. In the surface marker sorting group, both GABA neurons and 5-hydroxytryptamine neurons were significantly reduced to 1-2% in the graft (FIG. 19F). This result was further demonstrated by histological staining (FIGS. 20B-20E). Histological staining revealed that the GABA neurons and the 5-hydroxytryptamine neurons were also significantly reduced in the graft in the LMX1A and EN1 double-positive sorting group (FIG. 21B). The non-sorting group also included three different subtypes of glutamate neurons (a Glut_BARHL1+ cell population, a Glut_NKX2-1+ cell population, and a Glut_NKX6-1+ cell population), while the CLSTN2 or PTPRO sorting group included only one subtype of glutamate neurons (a Glut_NKX6-1+ cell population) (FIG. 19F). The representative marker for the two reduced cell populations Glut_BARHL1+ and Glut_NKX2-1+ was PITX2, which was considered a marker for diencephalon or lateral mFPs (Kirkeby et al., 2017; Nolbrant et al., 2017) (FIGS. 19D-19F). On the contrary, the Glut_NKX6-1+ cell population expressed the classic markers LMX1A, EN1, and FOXA2 of ventral mFPs. These data suggested that the Glut_NKX6-1+ cell populations might also be mFP-derived and had the same origin as mDA neurons (FIGS. 19E and 19F). Interestingly, the grafts sorted by different surface molecular markers had similar neuronal composition, which was mainly composed of three different subtypes of dopaminergic neurons (DA_0, DA_1, and DA_2) and one subtype of glutamatergic neurons (the Glut_NKX6-1+ cell population) (FIG. 19F). This result suggested that the compositions of grafts sorted by surface molecular markers were stable.

### Example 5 Evaluation of Innervation Patterns of Grafts

The innervation patterns of grafts were evaluated by staining human neuronal cell adhesion molecules (hNCAMs). In all sorting or non-sorting groups, the inventors observed dense hNCAM+ fibers (FIGS. 22A, 23A and 23B) covering the entire caudate putamen (CPu), which was a brain region targeted by endogenous DA neurons in the substantia nigra. Low-density hNCAM+ fibers were observed in target regions of endogenous mDA neurons in the lateral accumbens shell (LAcbSh) and olfactory tubercle (Tu) as well as ventral tegmental area (VTA) (FIGS. 22A, 23A, and 23B). Interestingly, the marker-sorted grafts were much smaller than the unsorted grafts, suggesting that sorting removed cells with high proliferative potential (FIGS. 22A and 22B).

Further examination revealed that STEM121-positive human nerve fibers (STEM121+) co-expressed TH in grafts from both sorting and non-sorting groups (FIG. 23D), demonstrating that these fibers were human dopaminergic nerve fibers derived from transplanted cells. In the marker-sorted grafts, human synaptophysin antibody-labeled punctate structures were distributed along TH-positive fibers in CPu, indicating that the transplanted mDA neurons formed synaptic junctions with host neurons (FIG. 23E).

To specifically set forth the specific innervation of the transplanted mDA neurons, the inventors constructed cell lines (TH-tdT/CLSTN2-EGFP hPSCs and TH-tdT/PTPRO-EGFP hPSCs), which expressed tdT via endogenous TH and expressed EGFP via CLSTN2 or PTPRO marker genes. TH-tdT hPSCs was taken as a control (FIG. 22C).

Then, surface marker sorted or unsorted neural precursor cells derived from these hPSC reporter lines were transplanted into the striatum of PD mice. Five months after transplantation, tdT was expressed only in the TH-positive mDA neurons in the grafts (FIG. 22D). Similar to the innervation pattern of hNCAM-positive fibers, tdT+ human mDA fibers from marker sorted or unsorted grafts were distributed throughout CPu (FIG. 22E). It was important that the tdT+ human mDA fibers from the marker sorted grafts were denser than those from the unsorted grafts, suggesting that the surface marker sorted grafts provided stronger dopaminergic innervation (FIGS. 22F and 22G).

### Example 6 Therapeutic Efficacy of CLSTN2- or PTPRO-Rich Neural Precursor Cells

6.1. The Electrophysiological Properties of Transplanted mDA Neurons were Examined.

Whole-cell patch-clamp recordings showed that human mDA neurons (tdT+) in sorted or unsorted grafts showed similar current-induced action potentials (APs) and spontaneous APs (sAPs) 5 months after transplantation, indicating that they were functionally mature (FIGS. 24A, 24B and 25A). Resting membrane potential (RMP) and AP thresholds were similar between sorting and non-sorting groups (FIGS. 24C and 24D). For all groups, sAPs showed low rating (unsorted, 0.39 Hz: CLSTN2, 0.91 Hz, PTPRO, 0.86 Hz; FIGS. 25A and 25B) and after hyperpolarization (AHP) protrusion (FIG. 24E). These features consistent with endogenous SNc (A9) mDA neurons. In addition, when injected with hyperpolarized currents, most transplanted mDA neurons in all groups showed sag potentials, which were typical features of A9 mDA neurons (FIGS. 25C and 25D). In addition, after reaching the maximum discharge frequency, the transplanted mDA neurons showed depolarization retardation, in response to the injection of increased gradient currents (FIGS. 24F and 24G). These results suggested that both sorted and unsorted mDA neurons were functionally mature 5 months after transplantation, and most of them had the electrophysiological characteristics of A9 mDA neurons.

The functional input of the transplanted mDA neurons was established 3-6 months after transplantation. In this example, the electrophysiological analysis of the transplanted mDA neurons showed that, 5 months after transplantation, spontaneous excitatory and inhibitory postsynaptic currents (sEPSC and sIPSC, respectively) were easily detected in the transplanted mDA neurons (FIG. 25E). The average amplitude and frequency of sIPSCs and sEPSCs were comparable between the sorting and non-sorting groups (FIGS. 25F, 25G, 24H and 4I). These results suggested that both surface marker-sorted and unsorted mDA neurons were incorporated into presynaptic circuits and received the functional input.

### 6.2. Evaluation of Functional Effects of Transplanted Cells

This example further evaluated the functional effects of the transplanted cells by amphetamine-induced rotation every 2 months before and after transplantation. PD mice transplanted with the neural precursor cells sorted or unsorted by CLSTN2, PTPRO, and LMX1A+EN1+ were gradually recovered during amphetamine-induced rotation (FIGS. 25H and 24J), while mice treated with the artificial cerebrospinal fluid (aCSF) alone did not recover (FIG. 25H). This result proved that the transplantation of both sorted and unsorted neural precursor cells could rescue the PD mice from behavioral disorders.

Based on this result and the fact that the marker-sorted cells were highly enriched with mDA neural precursor cells, it could be stated that, with transplantation of fewer marker-sorted cells described in the present invention, the functional recovery of PD mice should still be observed.

Hence, it was possible to reduce the number of transplanted cells per mouse from 100,000 to 7500 (i.e., less than 10% of the original graft dose). 6 months after transplantation, the recovery of amphetamine-induced rotational behavior was observed in the marker-sorted groups, but not in the unclassified control group, suggesting that the marker-sorted mDA neural precursor cells had a higher therapeutic efficacy (FIG. 25I).

The foregoing detailed description is provided by way of explanation and examples, and is not intended to limit the scope of the appended claims. Various changes of the embodiments listed in the present application until now would be obvious to those of ordinary skills in the art, and should be kept within the scope of the appended claims and equivalents thereof.

1. A method for identifying dopaminergic neural precursor cells, comprising:
determining whether candidate cells have one or more of the following features: CLSTN2⁺, PTPRO⁺, NTRK3⁺, FLRT2⁺, KITLG⁺, CD83⁺, and/or LMX1A⁺EN1⁺; and
identifying cells having said features as said dopaminergic neural precursor cells.

2. The method according to claim 1, wherein said candidate cells are neural precursor cells.

3. (canceled)

4. The method according to claim 1, wherein said candidate cells are derived from human pluripotent stem cells.

5-6. (canceled)

7. The method according to claim 1, wherein said determining comprises directly or indirectly detecting said candidate cells for expression and/or activity level of: CLSTN2 protein, PTPRO protein, NTRK3 protein, FLRT2 protein, KITLG protein, GD83 protein, and/or LMX1A protein and EN1 protein.

8. The method according to claim 7, wherein said expression and/or activity level of: CLSTN2 protein, PTPRO protein, NTRK3 protein, FLRT2 protein, KITLG protein, GD83 protein, and/or LMX1A protein and EN1 protein comprises:
expression and/or activity level of a nucleic acid molecule encoding: CLSTN2 protein, PTPRO protein, NTRK3 protein, FLRT2 protein, KITLG protein, GD83 protein, and/or LMX1A protein and EN1 protein; and/or expression and/or activity level of: CLSTN2 protein, PTPRO protein, NTRK3 protein, FLRT2 protein, KITLG protein, GD83 protein, and/or LMX1A protein and EN1 protein.

9. The method according to claim 7, wherein said detecting comprises modifying said candidate cells.

10. The method according to claim 7, wherein said candidate cells are detected with marker molecules.

11-12. (canceled)

13. The method according to claim 1, comprising bringing a reagent capable of specifically binding to: [said] CLSTN2 protein, PTPRO protein, NTRK3 protein, FLRT2 protein, KITLG protein, CD83 protein, and/or LMX1A protein and EN1 protein; and/or a reagent capable of measuring activity of: CLSTN2 protein, PTPRO protein, NTRK3 protein, FLRT2 protein, KITLG protein, CD83 protein, and/or LMX1A protein and EN1 protein into contact with said candidate cells.

14. The method according to claim 1, comprising bringing primers capable of specifically amplifying a nucleic acid molecule encoding: CLSTN2 protein, PTPRO protein, NTRK3 protein, FLRT2 protein, KITLG protein, CD83 protein, and/or LMX1A protein and EN1 protein; and/or probes capable of specifically recognizing said nucleic acid molecule encoding: CLSTN2 protein, PTPRO protein, NTRK3 protein, FLRT2 protein, KITLG protein, CD83 protein, and/or LMX1A protein and EN1 protein; into contact with said candidate cells.

15-71. (canceled)

72. A cell product, comprising dopaminergic neural precursor cells prepared according to claim 1.

73. A method for isolating and/or enriching dopaminergic neural precursor cells, comprising: (a) providing a neural precursor cell population, and (b) isolating and/or enriching, from said neural precursor cell population, cells having one or more of the following features: CLSTN2⁺, PTPRO⁺, NTRK3⁺, FLRT2⁺, KITLG⁺, CD83⁺, and/or LMX1A⁺EN1⁺.

74-77. (canceled)

78. A dopaminergic neural precursor cell population, comprising dopaminergic neural precursor cells prepared according to claim 73.

79. A method for preparing a cell product, comprising isolating and/or enriching dopaminergic neural precursor cells according to claim 73.

80-82. (canceled)

83. The method according to claim 79, wherein said cell population is derived from human pluripotent stem cells.

84-91. (canceled)

92. A method for evaluating a cell product or optimizing a preparation process for a cell product, comprising:
performing evaluation or optimization by detecting said cell product for a proportion of cells having one or more of the following features: CLSTN2⁺, PTPRO⁺, NTRK3⁺, FLRT2⁺, KITLG⁺, CD83⁺, and/or LMX1A⁺EN1⁺.

93. (canceled)

94. The method according to claim 92, wherein said preparation process comprises optimizing generation, differentiation, isolation, and/or purification of said cell product.

95. (canceled)

96. A method of preparing a cell product, the method comprising:
identifying an activity and/or level of a molecular marker with a CLSTN2+ indicator, a PTRO⁺ indicator, a NTRK3⁺ indicator, a FLRT2⁺ indicator, a KITLG⁺ indicator, a CD83⁺ indicator, and/or an LMX1A⁺EN1⁺ indicator,
wherein said cell product comprises dopaminergic neural precursor cells.

97-102. (canceled)

103. A quality control kit for preparing dopaminergic neural precursor cells, comprising a quality control reagent capable of determining whether candidate cells have one or more of the following features: CLSTN2⁺, PTPRO⁺, NTRK3⁺, FLRT2⁺, KITLG⁺, CD83⁺, and/or LMX1A⁺EN1⁺.

104. (canceled)

105. The quality control kit according to claim 103, wherein said candidate cells are neural precursor cells.

106. (canceled)

107. The quality control kit according to claim 103, wherein said candidate cells are derived from human pluripotent stem cells.

108. (canceled)

109. The quality control kit according to claim 103, wherein said reagent capable of culturing and/or preserving said candidate cells is packaged independently with said quality control reagent.

110. The quality control kit according to claim 103, wherein said quality control reagent is capable of directly or indirectly detecting said candidate cells for expression and/or activity level of: CLSTN2 protein, PTPRO protein, NTRK3 protein, FLRT2 protein, KITLG protein, CD83 protein, and/or LMX1A protein and EN1 protein.

111. The quality control kit according to claim 110, wherein said expression and/or activity level of: CLSTN2 protein, PTPRO protein, NTRK3 protein, FLRT2 protein, KITLG protein, CD83 protein, and/or LMX1A protein and EN1 protein comprises: expression and/or activity level of a nucleic acid molecule encoding: CLSTN2 protein, PTPRO protein, NTRK3 protein, FLRT2 protein, KITLG protein, CD83 protein, and/or LMX1A protein and EN1 protein, and/or expression and/or activity level of: CLSTN2 protein, PTPRO protein, NTRK3 protein, FLRT2 protein, KITLG protein, CD83 protein, and/or LMX1A protein and EN1 protein.

112-132. (canceled)

133. A method for controlling quality of prepared dopaminergic neural precursor cells, comprising:
a) detecting said prepared cells for a proportion of cells having one or more of the following features: CLSTN2⁺, PTPRO⁺, NTRK3⁺, FLRT2⁺, KITLG⁺, CD83⁺, and/or LMX1A⁺EN1⁺; and
b) if said proportion detected in a) is at least about 10%, determining that the quality of said prepared dopaminergic neural precursor cells is acceptable.

134-158. (canceled)

159. A cell product, comprising the dopaminergic neural precursor cell population according to claim 78.

160. A graft composition prepared from in vivo or in vitro differentiation of the dopaminergic neural precursor cell population according to claim 78.

161. A pharmaceutical composition, comprising the dopaminergic neural precursor cell population according to claim 78, and a pharmaceutically acceptable adjuvant.

162. (canceled)

163. A method for preventing, treating, or alleviating nervous system diseases or disorders, comprising administering the dopaminergic neural precursor cell population according to claim 78 to a subject in need thereof.

164. The method according to claim 163, wherein said nervous system diseases or disorders comprise Parkinson's disease.

165-168. (canceled)

169. A method for preventing, treating, or alleviating nervous system diseases or disorders, comprising:
identifying candidate cells for one or more of the following features: CLSTN2⁺, PTPRO⁺, NTRK3⁺, FLRT2⁺, KITLG⁺, CD83⁺, and/or LMX1A⁺EN1⁺;
selecting cells having said features; and
administering an effective dose of said cells having said features to a subject in need thereof.

170. The method according to claim 169, wherein said nervous system diseases or disorders comprise Parkinson's disease.
